(19) United States
(12) Patent Application Publication
(10) Pub. No.: US 2012/0143119 A1
(43) Pub. Date: Jun. 7, 2012

(54) Delivery of Serotonin Receptor Antagonists By Microinjection Systems

(75) Inventors: Anthony P. Deasey, Severna Park, MD (US); Patrick Frankham, Rosemere (CA); Wolfgang Renz, Beaconsfield (CA); Thomas Lang, Marshfield, MA (US)

(73) Assignee: LANCO Biosciences, Inc., Las Vegas, NV (US)

(21) Appl. No.: 13/294,895

(22) Filed: Nov. 11, 2011

Related U.S. Application Data
(60) Provisional application No. 61/419,173, filed on Dec. 2, 2010.

(51) Int. Cl. A61M 5/32; A61N 1/30; A61M 37/00

(52) U.S. Cl. 604/20; 604/272; 604/506; 604/22; 604/173

(57) ABSTRACT

In an aspect of the invention, serotonin receptor antagonist formulations are provided for subcutaneous, transdermal or intradermal delivery to a subject. In an embodiment, serotonin receptor antagonist formulations include ondansetron or ondansetron-containing formulations. In another embodiment, serotonin receptor antagonist formulations include palonosetron or palonosetron-containing formulations. In another aspect of the invention, microinjection devices are provided for delivering a serotonin receptor antagonist formulation to a subject.

### CROSS-REFERENCE

This applications claims priority to U.S. Provisional Patent Application Ser. No. 61/419,173, filed Dec. 2, 2010, which is entirely incorporated herein by reference.

### BACKGROUND OF THE INVENTION

Serotonin receptors, also known as 5-hydroxytryptamine receptors (or 5-HT receptors), are a group of G protein-coupled receptors (GPCRs) and ligand-gated ion channels (LGICs) found in human central and peripheral nervous systems. Serotonin receptors can mediate excitatory and inhibitory neurotransmission. Serotonin receptors are typically activated by serotonin, a neurotransmitter that acts as a natural ligand. Serotonin receptors can modulate the release of various neurotransmitters, including glutamate, GABA, dopamine, epinephrine, norepinephrine, and acetylcholine, in addition to various hormones, such as oxytocin, prolactin, vasopressin, cortisol, corticotropin and substance P. The serotonin receptors can influence various biological and neurological processes.

5-HT₃ antagonists are a class of medications that act as receptor antagonists at the 5-HT₃ receptor (5-HT₃ receptor), a subtype of serotonin receptor found in terminals of the vagus nerve and in certain areas of the human brain. 5-HT₃ antagonists can be antiemetics, used in the prevention and treatment of nausea and vomiting. They can be effective in controlling the nausea and vomiting produced by cancer chemotherapy. 5-HT₃ antagonist include tropisetron, granisetron, ondansetron, palonosetron, dolasetron, metocclopromide and benzoylmethylecgonine.

Ondansetron (trade name Zofran®) is a serotonin 5-HT₃ receptor antagonist that can be used as an antiemetic to treat nausea and vomiting during the course of chemotherapy. Ondansetron can reduce the activity of the vagus nerve, which can deactivate the vomiting center in the medulla oblongata and block serotonin receptors in the chemoreceptor trigger zone. Palonosetron (trade name Aloxi®) is a serotonin 5-HT₃ antagonist that can be used in the prevention and treatment of chemotherapy-induced nausea and vomiting.

### SUMMARY OF THE INVENTION

In an aspect of the invention, a system comprises a microinjection device and a serotonin receptor antagonist formulation, the microinjection device comprising a microneedle array having one or more hollow tips for delivering a serotonin receptor antagonist formulation; a housing having the microneedle array and a skin-contacting face defining an opening that can be positioned at or adjacent to a target site; and a driver for moving the microneedle array toward the target site. In an embodiment, the serotonin receptor antagonist formulation has a pH between about 2.0 and 6.0. In another embodiment, the serotonin receptor antagonist formulation has a pH between about 3.0 and 5.9. In another embodiment, the serotonin receptor antagonist formulation has a pH between about 3.1 and 5.8. In another embodiment, the serotonin receptor antagonist formulation has a pH between about 3.3 and 5.5. In another embodiment, the serotonin receptor antagonist formulation comprises ondansetron, palonosetron, tropisetron, granisetron, dolasetron, metoclopramide, benzoylmethylecgonine, or a pharmaceutically acceptable derivative thereof. In another embodiment, the serotonin receptor antagonist formulation has a serotonin receptor antagonist concentration between about 0.001 mg in 1 milliliter (mL) and 40 mg in 1 mL. In another embodiment, the serotonin receptor antagonist concentration is between about 0.01 mg in 1 mL and 30 mg in 1 mL.

In another aspect, a method for delivering a serotonin receptor antagonist to a subject comprises providing a microinjection device comprising a microneedle array and a serotonin receptor antagonist-containing formulation; and delivering the serotonin receptor antagonist-containing formulation to the subject with the aid of the microinjection device. In an embodiment, the serotonin receptor antagonist formulation comprises ondansetron, palonosetron, tropisetron, granisetron, dolasetron, metoclopramide, benzoylmethylecgonine, or a pharmaceutically acceptable derivative thereof. In another embodiment, the serotonin receptor antagonist formulation has a serotonin receptor antagonist concentration between about 0.001 mg in 1 milliliter (mL) and 40 mg in 1 mL. In another embodiment, the serotonin receptor antagonist concentration is between about 0.01 mg in 1 mL and 30 mg in 1 mL. In another embodiment, the serotonin receptor antagonist formulation has a pH between about 2.0 and 6.0. In another embodiment, the serotonin receptor antagonist formulation has a pH between about 3.0 and 5.9. In another embodiment, the serotonin receptor antagonist formulation has a pH between about 3.1 and 5.8. In another embodiment, the serotonin receptor antagonist formulation has a pH between about 3.3 and 5.5.

In another aspect of the invention, a method for treating nausea or vomiting in a subject comprises using a microinjection device comprising a microneedle array and a serotonin receptor antagonist formulation to administer to the subject the serotonin receptor antagonist formulation. In an embodiment, the serotonin receptor antagonist formulation comprises ondansetron, palonosetron, tropisetron, granisetron, dolasetron, metoclopramide, benzoylmethylecgonine, or a pharmaceutically acceptable derivative thereof. In another embodiment, the serotonin receptor antagonist formulation is administered to the subject on a daily basis. In another embodiment, the serotonin receptor antagonist formulation has a serotonin receptor antagonist concentration between about 0.001 mg in 1 milliliter (mL) and 40 mg in 1 mL. In another embodiment, the serotonin receptor antagonist concentration is between about 0.01 mg in 1 mL and 30 mg in 1 mL. In another embodiment, the serotonin receptor antagonist formulation has a pH between about 2.0 and 6.0. In another embodiment, the serotonin receptor antagonist formulation has a pH between about 3.0 and 5.9. In another embodiment, the serotonin receptor antagonist formulation has a pH between about 3.1 and 5.8. In another embodiment, the serotonin receptor antagonist formulation has a pH between about 3.3 and 5.5. In another embodiment, the serotonin receptor antagonist formulation is delivered to the subject in a length of time between about 0.1 seconds and 10 minutes. In another embodiment, the serotonin receptor antagonist formulation is delivered to the subject in a length of time between about 30 seconds and 8 minutes. In another embodiment, the serotonin receptor antagonist formulation is delivered transdermally. In another embodiment, the serotonin receptor antagonist formulation is delivered intradermally. In another embodiment, the serotonin receptor antagonist formulation is delivered subcutaneously.

In another aspect of the invention, a system comprising an application device and a serotonin receptor antagonist formulation is provided. The application device comprises a housing having a skin-contacting face defining an opening that can be positioned at a target site, the housing having a microneedle array; and an impactor for impacting the microneedle array and accelerating the microneedle array toward the target site, the microneedle array configured to deliver the serotonin receptor antagonist formulation to the subject. The impactor is configured to move along a substantially arcuate path to move the microneedle array toward the target site. In an embodiment, the serotonin receptor antagonist formulation comprises ondansetron, palonosetron, tropisetron, granisetron, dolasetron, metoclopramide, benzoylmethylecgonine, or a pharmaceutically acceptable derivative thereof. In another embodiment, the serotonin receptor antagonist formulation has a serotonin receptor antagonist concentration between about 0.001 mg in 1 milliliter (mL) and 40 mg in 1 mL. In another embodiment, the serotonin receptor antagonist concentration is between about 0.01 mg in 1 mL and 30 mg in 1 mL. In another embodiment, the serotonin receptor antagonist formulation has a pH between about 2.0 and 6.0. In another embodiment, the serotonin receptor antagonist formulation has a pH between about 3.0 and 5.9. In another embodiment, the serotonin receptor antagonist formulation has a pH between about 3.1 and 5.8. In another embodiment, the serotonin receptor antagonist formulation has a pH between about 3.3 and 5.5.

In another aspect of the invention, a microinjection device having a serotonin receptor antagonist formulation is provided. The microinjection device is configured to deliver the serotonin receptor antagonist formulation to a subject. In an embodiment, the serotonin receptor antagonist formulation comprises ondansetron, palonosetron, tropisetron, granisetron, dolasetron, metoclopramide, benzoylmethylecgonine, or a pharmaceutically acceptable derivative thereof. In another embodiment, the serotonin receptor antagonist formulation has a serotonin receptor antagonist concentration between about 0.001 mg in 1 milliliter (mL) and 40 mg in 1 mL. In another embodiment, the serotonin receptor antagonist concentration is between about 0.01 mg in 1 mL and 30 mg in 1 mL. In another embodiment, the serotonin receptor antagonist formulation has a pH between about 2.0 and 6.0. In another embodiment, the serotonin receptor antagonist formulation has a pH between about 3.0 and 5.9. In another embodiment, the serotonin receptor antagonist formulation has a pH between about 3.1 and 5.8. In another embodiment, the serotonin receptor antagonist formulation has a pH between about 3.3 and 5.5.

In another aspect of the invention, a microinjection device for subcutaneous, transdermal or intradermal delivery of a serotonin receptor antagonist formulation to a subject comprises a microneedle array for delivering a serotonin receptor antagonist formulation to a subject; and one or more chambers in fluid communication with the microneedle array, the one or more chambers having a serotonin receptor antagonist formulation. In an embodiment, the microneedle array comprises microneedles having hollow tips. In another embodiment, the serotonin receptor antagonist formulation comprises ondansetron, palonosetron, tropisetron, granisetron, dolasetron, metoclopramide, benzoylmethylecgonine, or a pharmaceutically acceptable derivative thereof. In another embodiment, the serotonin receptor antagonist formulation has a serotonin receptor antagonist concentration between about 0.001 mg in 1 milliliter (mL) and 40 mg in 1 mL. In another embodiment, the serotonin receptor antagonist concentration is between about 0.01 mg in 1 mL and 30 mg in 1 mL. In another embodiment, the serotonin receptor antagonist formulation has a pH between about 2.0 and 6.0. In another embodiment, the serotonin receptor antagonist formulation has a pH between about 3.0 and 5.9. In another embodiment, the serotonin receptor antagonist formulation has a pH between about 3.1 and 5.8. In another embodiment, the serotonin receptor antagonist formulation has a pH between about 3.3 and 5.5.

In another aspect of the invention, a system for the administration of a serotonin receptor antagonist to a subject comprises a serotonin receptor antagonist formulation; and a microinjection device. In an embodiment, the serotonin receptor antagonist formulation is ondansetron, palonosetron, tropisetron, granisetron, dolasetron, metoclopramide, benzoylmethylecgonine, or a pharmaceutically acceptable derivative thereof. In another embodiment, the serotonin receptor antagonist formulation has a serotonin receptor antagonist concentration between about 0.001 mg in 1 milliliter (mL) and 40 mg in 1 mL. In another embodiment, the serotonin receptor antagonist concentration is between about 0.01 mg in 1 mL and 30 mg in 1 mL. In another embodiment, the serotonin receptor antagonist formulation has a pH between about 2.0 and 6.0. In another embodiment, the serotonin receptor antagonist formulation has a pH between about 3.0 and 5.9. In another embodiment, the serotonin receptor antagonist formulation has a pH between about 3.1 and 5.8. In another embodiment, the serotonin receptor antagonist formulation has a pH between about 3.3 and 5.5.

In another aspect of the invention, a system for applying a microneedle array to a subject's skin comprises a serotonin receptor antagonist formulation; a housing having a skin-contacting face defining an opening that can be positioned at a target site, the housing having a microneedle array; and an impactor for impacting the microneedle array and accelerating the microneedle array toward the target site, the microneedle array configured to deliver a serotonin receptor antagonist formulation to the subject. The impactor is configured to move along a substantially arcuate path to move the microneedle array toward the target site. In an embodiment, the serotonin receptor antagonist formulation comprises ondansetron, palonosetron, tropisetron, granisetron, dolasetron, metoclopramide, benzoylmethylecgonine, or a pharmaceutically acceptable derivative thereof. In another embodiment, the serotonin receptor antagonist formulation has a serotonin receptor antagonist concentration between about 0.001 mg in 1 milliliter (mL) and 40 mg in 1 mL. In another embodiment, the serotonin receptor antagonist concentration is between about 0.01 mg in 1 mL and 30 mg in 1 mL. In another embodiment, the serotonin receptor antagonist formulation has a pH between about 2.0 and 6.0. In another embodiment, the serotonin receptor antagonist formulation has a pH between about 3.0 and 5.9. In another embodiment, the serotonin receptor antagonist formulation has a pH between about 3.1 and 5.8. In another embodiment, the serotonin receptor antagonist formulation has a pH between about 3.3 and 5.5.

In an aspect of the invention, a system for subcutaneous, transdermal or intradermal delivery of a serotonin receptor antagonist to a subject comprises a serotonin receptor antagonist formulation; a microneedle array for delivering the serotonin receptor antagonist formulation to a subject; and one or more chambers in fluid communication with the microneedle array, the one or more chambers configured to hold the serotonin receptor antagonist formulation. In an embodiment, the serotonin receptor antagonist formulation comprises ondansetron, palonosetron, tropisetron, granisetron, dolasetron, metoclopramide, benzoylmethylecgonine, or a pharmaceutically acceptable derivative thereof. In another embodiment, the serotonin receptor antagonist formulation has a serotonin receptor antagonist concentration between about 0.001 mg in 1 mL and 40 mg in 1 mL. In another embodiment, the serotonin receptor antagonist formulation has a serotonin receptor antagonist concentration between about 0.01 mg in 1 mL and 30 mg in 1 mL. In another embodiment, the serotonin receptor antagonist formulation has a pH between about 2.0 and 6.0. In another embodiment, the serotonin receptor antagonist formulation has a pH between about 3.0 and 5.9. In another embodiment, the serotonin receptor antagonist formulation has a pH between about 3.1 and 5.8. In another embodiment, the serotonin receptor antagonist formulation has a pH between about 3.3 and 5.5.

In another aspect of the invention, a system for delivering a serotonin receptor antagonist formulation to a subject comprises a serotonin receptor antagonist formulation; a microneedle array having one or more hollow tips for delivering the serotonin receptor antagonist formulation; a housing having the microneedle array and a skin-contacting face defining an opening that can be positioned at or adjacent to a target site; and a driver for moving the microneedle array toward the target site. In an embodiment, the serotonin receptor antagonist formulation has a serotonin receptor antagonist concentration between about 0.001 mg in 1 milliliter (mL) and 40 mg in 1 mL. In another embodiment, the serotonin receptor antagonist concentration is between about 0.01 mg in 1 mL and 30 mg in 1 mL. In another embodiment, the serotonin receptor antagonist formulation comprises ondansetron, palonosetron, tropisetron, granisetron, dolasetron, metoclopramide, benzoylmethylecgonine, or a pharmaceutically acceptable derivative thereof. In another embodiment, the serotonin receptor antagonist formulation has a pH between about 2.0 and 6.0. In another embodiment, the serotonin receptor antagonist formulation has a pH between about 3.0 and 5.9. In another embodiment, the serotonin receptor antagonist formulation has a pH between about 3.1 and 5.8. In another embodiment, the serotonin receptor antagonist formulation has a pH between about 3.3 and 5.5.

In another aspect of the invention, a microinjection device comprises a hollow microneedle array and a serotonin receptor antagonist formulation, the microinjection device configured to deliver the serotonin receptor antagonist formulation to a subject. In an embodiment, the serotonin receptor antagonist formulation comprises ondansetron, palonosetron, tropisetron, granisetron, dolasetron, metoclopramide, benzoylmethylecgonine, or a pharmaceutically acceptable derivative thereof. In another embodiment, the serotonin receptor antagonist formulation has a serotonin receptor antagonist concentration between about 0.001 mg in 1 milliliter (mL) and 40 mg in 1 mL. In another embodiment, the serotonin receptor antagonist concentration is between about 0.01 mg in 1 mL and 30 mg in 1 mL. In another embodiment, the serotonin receptor antagonist formulation has a pH between about 2.0 and 6.0. In another embodiment, the serotonin receptor antagonist formulation has a pH between about 3.0 and 5.9. In another embodiment, the serotonin receptor antagonist formulation has a pH between about 3.1 and 5.8. In another embodiment, the serotonin receptor antagonist formulation has a pH between about 3.3 and 5.5.

In another aspect of the invention, a method for subcutaneous, transdermal or intradermal delivery of a serotonin receptor antagonist formulation to a subject comprises administering the serotonin receptor antagonist formulation to the subject with ion pairs, coacervates, vesicles, liposomes, or particles. In an embodiment, the serotonin receptor antagonist formulation comprises ondansetron, palonosetron, tropisetron, granisetron, dolasetron, metoclopramide, benzoylmethylecgonine, or a pharmaceutically acceptable derivative thereof. In another embodiment, the serotonin receptor antagonist formulation has a serotonin receptor antagonist concentration between about 0.001 mg in 1 milliliter (mL) and 40 mg in 1 mL. In another embodiment, the serotonin receptor antagonist concentration is between about 0.01 mg in 1 mL and 30 mg in 1 mL. In another embodiment, the serotonin receptor antagonist formulation has a pH between about 2.0 and 6.0. In another embodiment, the serotonin receptor antagonist formulation has a pH between about 3.0 and 5.9. In another embodiment, the serotonin receptor antagonist formulation has a pH between about 3.1 and 5.8. In another embodiment, the serotonin receptor antagonist formulation has a pH between about 3.3 and 5.5. In another embodiment, particles are used to administer the serotonin receptor antagonist formulation. In another embodiment, the particles are administered to a subject's skin at a high velocity.

In another aspect of the invention, a method for subcutaneous, transdermal or intradermal delivery of a serotonin receptor antagonist formulation to a subject comprises administering the serotonin receptor antagonist formulation to the subject by microneedle injection, hydration, ablation of the subject's skin, follicular delivery, ultrasound, iontophoresis or electroporation. In an embodiment, the serotonin receptor antagonist formulation comprises ondansetron, palonosetron, tropisetron, granisetron, dolasetron, metoclopramide, benzoylmethylecgonine, or a pharmaceutically acceptable derivative thereof. In another embodiment, the serotonin receptor antagonist formulation has a serotonin receptor antagonist concentration between about 0.001 mg in 1 milliliter (mL) and 40 mg in 1 mL. In another embodiment, the serotonin receptor antagonist formulation has a serotonin receptor antagonist concentration between about 0.01 mg in 1 mL and 30 mg in 1 mL. In another embodiment, the serotonin receptor antagonist formulation has a pH between about 2.0 and 6.0. In another embodiment, the serotonin receptor antagonist formulation has a pH between about 3.0 and 5.9. In another embodiment, the serotonin receptor antagonist formulation has a pH between about 3.1 and 5.8. In another embodiment, the serotonin receptor antagonist formulation has a pH between about 3.3 and 5.5. In another embodiment, the serotonin receptor antagonist formulation is administered to the subject by microneedle injection. In another embodiment, the serotonin receptor antagonist formulation is administered to the subject by iontophoresis.

In another aspect of the invention, a method for treating nausea or vomiting induced by chemotherapy comprises using a microinjection device comprising a microneedle array and a serotonin receptor antagonist formulation to administer to a subject the serotonin receptor antagonist formulation. In an embodiment, the serotonin receptor antagonist formulation comprises ondansetron, palonosetron, tropisetron, granisetron, dolasetron, metoclopramide, benzoylmethylecgonine, or a pharmaceutically acceptable derivative thereof. In another embodiment, the serotonin receptor antagonist formulation is administered to the subject on a daily basis. In another embodiment, the serotonin receptor antagonist formulation has a serotonin receptor antagonist concentration between about 0.001 mg in 1 milliliter (mL) and 40 mg in 1 mL. In another embodiment, the serotonin receptor antagonist concentration is between about 0.01 mg in 1 mL and 30 mg in 1 mL. In another embodiment, the serotonin receptor antagonist formulation has a pH between about 2.0 and 6.0. In another embodiment, the serotonin receptor antagonist formulation has a pH between about 3.0 and 5.9. In another embodiment, the serotonin receptor antagonist formulation has a pH between about 3.1 and 5.8. In another embodiment, the serotonin receptor antagonist formulation has a pH between about 3.3 and 5.5. In another embodiment, the serotonin receptor antagonist formulation is delivered to the subject in a length of time between about 0.1 seconds and 10 minutes. In another embodiment, the serotonin receptor antagonist formulation is delivered to the subject in a length of time between about 30 seconds and 8 minutes. In another embodiment, the serotonin receptor antagonist formulation is delivered transdermally. In another embodiment, the serotonin receptor antagonist formulation is delivered intradermally. In another embodiment, the serotonin receptor antagonist formulation is delivered subcutaneously.

In another aspect of the invention, a system for delivering a serotonin receptor antagonist formulation to a subject comprises a serotonin receptor antagonist formulation having an excipient; and a microinjection device configured to deliver the serotonin receptor antagonist formulation. In an embodiment, the serotonin receptor antagonist formulation comprises ondansetron, palonosetron, tropisetron, granisetron, dolasetron, metoclopramide, benzoylmethylecgonine, or a pharmaceutically acceptable derivative thereof. In another embodiment, the serotonin receptor antagonist formulation has a serotonin receptor antagonist concentration between about 0.001 mg in 1 milliliter (mL) and 40 mg in 1 mL. In another embodiment, the serotonin receptor antagonist concentration is between about 0.01 mg in 1 mL and 30 mg in 1 mL. In another embodiment, the serotonin receptor antagonist formulation has a pH between about 2.0 and 6.0. In another embodiment, the serotonin receptor antagonist formulation has a pH between about 3.0 and 5.9. In another embodiment, the serotonin receptor antagonist formulation has a pH between about 3.1 and 5.8. In another embodiment, the serotonin receptor antagonist formulation has a pH between about 3.3 and 5.5. In another embodiment, the microinjection device is configured to deliver at least about 0.001 mg or 0.01 mg of the serotonin receptor antagonist formulation in 1 mL of the serotonin receptor antagonist formulation. In another embodiment, the serotonin receptor antagonist formulation comprises ondansetron or a pharmaceutically acceptable derivative of ondansetron. In another embodiment, the excipient includes one or more of citric acid anhydrous, sodium benzoate, sodium citrate, sorbitol and water. In another embodiment, the serotonin receptor antagonist formulation comprises palonosetron or a pharmaceutically acceptable derivative of palonosetron. In another embodiment, the excipient includes one or more of mannitol, disodium edetate, citrate and water. In another embodiment, the excipient is a pharmaceutically acceptable excipient.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is a perspective view of a microinjection device having a microneedle assembly, in accordance with an embodiment of the invention; FIG. 1B is a perspective side view of an array of microneedles, in accordance with an embodiment of the invention;

FIG. 2 is a schematic cross-sectional side view of a microinjection device having an array of microneedles, in accordance with an embodiment of the invention;

FIG. 3 is a schematic cross-sectional side view of a portion of the microinjection device of FIG. 2, in accordance with an embodiment of the invention;

FIG. 4 is a schematic perspective side view of a microneedle device comprising a patch, in accordance with an embodiment of the invention;

FIG. 5A is a perspective side view of an array of microneedles, in accordance with an embodiment of the invention. FIG. 5B is a cross-sectional side view of a microneedle in the array of FIG. 5A, in accordance with an embodiment of the invention;

FIG. 6 is a schematic side view of a microneedle application device, in accordance with an embodiment of the invention;

FIG. 7 is a schematic cross sectional side view of the microneedle application device of FIG. 6, in accordance with an embodiment of the invention;

FIG. 8 is a schematic cross sectional side view of a collar of the microneedle application device of FIGS. 6 and 7, in accordance with an embodiment of the invention;

FIG. 9A is a schematic perspective view of an applicator device having peelable seals, in accordance with an embodiment of the invention. FIG. 9B is a schematic perspective view of the applicator of FIG. 9A with the peelable seals removed, in accordance with an embodiment of the invention. FIG. 9C is a schematic cross-sectional view of the applicator of FIGS. 9A and 9B in a loaded position, in accordance with an embodiment of the invention. FIG. 9D is a schematic cross-sectional view of the applicator of FIGS. 9A and 9B in a partially released position, in accordance with an embodiment of the invention. FIG. 9E is a schematic cross-sectional view of the applicator of FIGS. 9A and 9B in a position where a microneedle array can contact a target surface, in accordance with an embodiment of the invention. FIG. 9F is a schematic cross-sectional view of the applicator of FIGS. 9A and 9B being removed from a microneedle array that has been deployed onto a target surface, in accordance with an embodiment of the invention;

FIG. 10 is a schematic cross-sectional side view of an applicator device, in accordance with an embodiment of the invention;

FIG. 11 is a schematic perspective view of a portion of the applicator device of FIG. 10, in accordance with an embodiment of the invention;

FIG. 12 is a schematic perspective view of an applicator device having a patch, in accordance with an embodiment of the invention; and

FIG. 13 is a schematic partial cross-sectional side view of a microneedle array cartridge mounted on an applicator device, in accordance with an embodiment of the invention.

### DETAILED DESCRIPTION OF THE INVENTION

While preferable embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention.

The term “serotonin receptor antagonist,” as used herein, can include any species or compound, including a formulation having a species or compound, configured to reduce or eliminate nausea, vomiting or both nausea and vomiting, such as nausea and/or vomiting induced by chemotherapy. In another embodiment, serotonin receptor antagonists can include species or compounds that are configured to treat, reduce, or eliminate schizophrenia, sleep apnea, Parkinson's disease, obsessive compulsive disorder, alcoholism, opioid addiction, irritable bowel syndrome and postanesthetic shivering. In embodiments, serotonin receptor antagonists can include serotonin 5-HT₃ receptor antagonists. In some embodiments, serotonin receptor antagonists include ondansetron, palonosetron, tropisetron, granisetron, dolasetron, metoclopramide, benzoylmethylecgonine, or salts or pharmaceutically acceptable derivative thereofs. In an embodiment, serotonin receptor antagonists can include ondansetron, having the chemical formula C₁₈H₁₉N₃O and systematic name (RS)-9-methyl-3-[(2-methyl-1H-imidazol-1-yl)methyl]-2,3-dihydro-1H-carbazol-4(9H)-one, a variant or derivative of ondansetron, or a pharmaceutically acceptable variant or derivative of ondansetron. In another embodiment, serotonin receptor antagonists can include ondansetron hydrochloride (HCl) dihydrate. In another embodiment, serotonin receptor antagonists can include palonosetron, having the chemical formula and systematic name (3aR)-2-[(3S)-1-azabicyclo[2.2.2]oct-3-yl]-2,3,3a,4,5,6-hexahydro-1H-benz[de]isoquinolin-1-one, a variation of palonosetron, or a pharmaceutically acceptable variant or derivative of palonosetron. In another embodiment, serotonin receptor antagonists can include palonosetron HCl. A serotonin receptor antagonist can include other medically active or inactive species, compounds, or formulations.

Serotonin receptor antagonists disclosed herein can be provided as salts thereof, such as, without limitation, ammonium salts (or derivatives), nitrate salts, nitrite salts, phosphate salts, sulfate salts, carbonate salts, or hydrochloride salts.

The terms “transdermal” and “transdermally,” as used herein, can refer to transdermal drug delivery. In an embodiment, transdermal drug delivery can include delivering a drug or formulation to a subject across the subject's skin. In another embodiment, transdermal drug delivery can include delivering a drug or formulation to a subject across the subject's skin and into a blood vessel.

The terms “intradermal” and “intradermally,” as used herein, can refer to intradermal drug delivery. In an embodiment, intradermal drug delivery can include delivering a drug or formulation to a subject in or into the subject's skin.

The term “subcutaneous injection,” as used herein, can refer to an injection that is administered as a bolus into the subcutis, i.e., the layer of skin directly below the dermis and epidermis (collectively referred to as the cutis).

### Serotonin Receptor Antagonist Compounds and Formulations

In an aspect of the invention, serotonin receptor antagonist compounds (also “serotonin receptor antagonists” herein) are provided that can be used to treat nausea and/or vomiting, schizophrenia, sleep apnea, Parkinson's disease, obsessive compulsive disorder, alcoholism, opioid addiction, irritable bowel syndrome and postanesthetic shivering. In an embodiment, serotonin receptor antagonists include ondansetron or pharmaceutically acceptable derivatives of ondansetron.

In an embodiment an injection solution comprising a serotonin receptor antagonist compound can be filled into primary packaging, such as an injection or microinjection apparatuses, as described below. Serotonin receptor antagonist compounds and formulations of embodiments of the invention can be administered to one or more subjects with the aid of injection or microinjection apparatuses of embodiments of the invention.

In an embodiment, a serotonin receptor antagonist compound, such as an ondansetron compound, can be included in a formulation that further comprises other compounds or agents, such as other pharmacological agents.

In an embodiment, serotonin receptor antagonist compounds are provided that can reduce the activity of the vagus nerve. In another embodiment, ondansetron or an ondansetron formulation is provided for reducing the activity of the vagus nerve. This can advantageously reduce nausea and/or vomiting, such as nausea and/or vomiting induced by chemotherapy. In some cases, this can help reduce or treat schizophrenia, sleep apnea, Parkinson's disease, obsessive compulsive disorder, alcoholism, opioid addiction, irritable bowel syndrome and postanesthetic shivering.

In an embodiment, a serotonin receptor antagonist compound comprises a serotonin 5-HT₃ receptor antagonist compound. In another embodiment, a serotonin receptor antagonist compound can include ondansetron (or ondansetron base), having the formula C₁₈H₁₉N₃O, and the following structure:

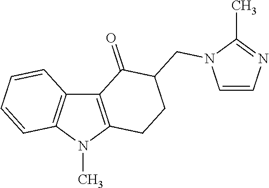

Serotonin receptor antagonists can be coordinated to other compounds, such as water, acids, alcohols and bases. In an embodiment, a serotonin receptor antagonists can include ondansetron hydrochloride (HCl) dihydrate, which can have the following structure:

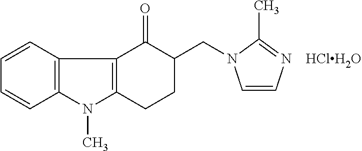

In another embodiment, a serotonin receptor antagonist compound can include palonosetron (or palonosetron base), having the formula C₁₉H₂₄N₂O, and the following structure:

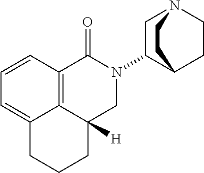

In an embodiment, a serotonin receptor antagonists can include palonosetron hydrochloride (HCl), which can have the following structure:

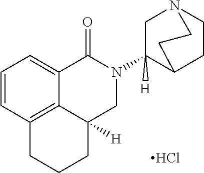

In another embodiment, a serotonin receptor antagonist compound can include tropisetron, having the formula C₁₇H₂₀N₂O₂, and the following structure:

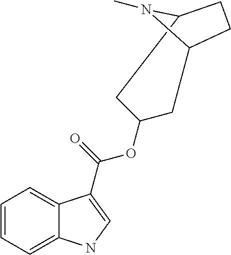

In another embodiment, a serotonin receptor antagonist compound can include granisetron, having the formula C₁₈H₂₄N₄O, and the following structure:

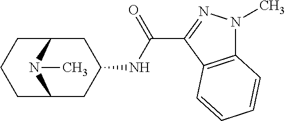

In another embodiment, a serotonin receptor antagonist compound can include dolasetron, having the formula C₁₉H₂₀N₂O₃, and the following structure:

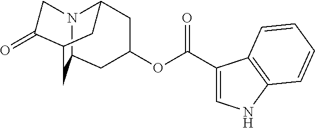

In another embodiment, a serotonin receptor antagonist compound can include metoclopramide, having the formula C₁₄H₂₂ClN₃O₂, and the following structure:

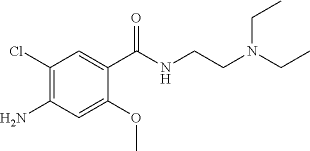

In another embodiment, a serotonin receptor antagonist compound can include benzoylmethylecgonine, having the formula C₁₇H₂₁NO₄, and the following structure:

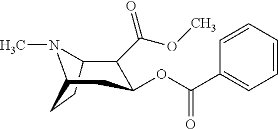

A serotonin receptor antagonist-containing formulation can be configured for one or more of subcutaneous delivery, intradermal delivery and transdermal delivery to a subject (e.g., patient). In an embodiment, a serotonin receptor antagonist-containing formulation can be delivered to a subject with the aid of microinjection or microneedle devices, as described below.

In an embodiment, a serotonin receptor antagonist compound can include ondansetron (or ondansetron base) having the empirical formula C₁₈H₁₉N₃O, representing a molecular weight of about 293.4 g/mol. In another embodiment, a serotonin receptor antagonist compound can include an ondansetron acid, such as ondansetron HCl dihydrate and having the empirical formula C₁₈H₁₉N₃O.HCl.2H₂O, representing a molecular weight of about 365.9 g/mol. In another embodiment, a serotonin receptor antagonist compound can include a palonosetron compound, having the chemical formula C₁₉H₂₄N₂O, representing a molecular weight of about 296.407 g/mol. In another embodiment, a serotonin receptor antagonist compound can include a palonosetron acid, such as palonosetron HCl and having the chemical formula C₁₉H₂₄N₂O.HCl, representing a molecular weight of about 332.87 g/mol.

In an embodiment, an serotonin receptor antagonist compound can be a white to off-white substance, such as a powder, that is readily soluble in water and in a saline solution. In another embodiment, an ondansetron or ondansetron-containing compound (e.g., ondansetron base, ondansetron acid) can be a white to off-white substance, such as a powder, that is readily soluble in water and in a saline solution. In another embodiment, a palonosetron compound or palonosetron-containing compound (e.g., palonosetron acid) can be a white to off-white substance, such as a crystalline powder, that is freely soluble in water, soluble in propylene glycol, and slightly soluble in ethanol and 2-propanol.

In an embodiment, a serotonin receptor antagonist formulation can have a pH between about 2.0 and 6.0, or between about 3.0 and 5.9, or between about 3.1 and 5.8, or between about 3.3 and 5.5. In another embodiment, a serotonin receptor antagonist formulation can have a pH of about 2.0, or 2.1, or 2.2, or 2.3, or 2.4, or 2.5, or 2.6, or 2.7, or 2.8, or 2.9, or 3.0, or 3.1, or 3.2, or 3.3, or 3.4, or 3.5, or 3.6, or 3.7, or 3.8, or 3.9, or 4.0, or 4.1, or 4.2, or 4.3, or 4.4, or 4.5, or 4.6, or 4.7, or 4.8, or 4.9, or 5.0, or 5.1, or 5.2, or 5.3, or 5.4, or 5.5, or 5.6, or 5.7, or 5.8, or 5.9, or 6.0.

In an embodiment, an ondansetron-containing formulation can have a pH between about 2.0 and 6.0, or between about 3.0 and 5.5, or between about 3.1 and 4.5, or between about 3.3 and 4.0. In another embodiment, an ondansetron-containing formulation can have a pH of about 2.0, or 2.1, or 2.2, or 2.3, or 2.4, or 2.5, or 2.6, or 2.7, or 2.8, or 2.9, or 3.0, or 3.1, or 3.2, or 3.3, or 3.4, or 3.5, or 3.6, or 3.7, or 3.8, or 3.9, or 4.0, or 4.1, or 4.2, or 4.3, or 4.4, or 4.5.

In an embodiment, a palonosetron-containing formulation can have a pH between about 2.0 and 6.0, or between about 3.0 and 5.9, or between about 4.0 and 5.8, or between about 4.5 and 5.5. In another embodiment, a palonosetron-containing formulation can have a pH of about 4.0, or 4.1, or 4.2, or 4.3, or 4.4, or 4.5, or 4.6, or 4.7, or 4.8, or 4.9, or 5.0, or 5.1, or 5.2, or 5.3, or 5.4, or 5.5, or 5.6, or 5.7, or 5.8, or 5.9, or 6.0.

A serotonin receptor antagonist formulation can be lyophilized and formed into an aqueous solution suitable for subcutaneous, transdermal or intradermal injection. Alternatively, a serotonin receptor antagonist formulation can be formulated in any of the forms known in the art for preparing oral, nasal, buccal, or rectal formulations of peptide drugs.

Serotonin receptor antagonist formulations, such as ondansetron-containing formulations or palonosetron-containing formulations, can be combined or modified with various substances or components, including, without limitation, glidants, lubricants, antioxidants, antimicrobial agents, enzyme inhibitors, stabilizers (including pH stabilizers), retarding agents, preservatives and modifiers.

Serotonin receptor antagonist formulations, including ondansetron-containing formulations and palonosetron-containing formulations, can include other pharmaceutically active or inactive ingredients. Such ingredients can be added to provide a desirable fluid property of the formulation, such as a desirable viscosity for administering the formulation using a microinjection device. In an embodiment, a serotonin receptor antagonist formulation can include one or more excipients (inactive ingredients), such as dyes, flavors, binders, emollients, fillers, lubricants and preservatives. In another embodiment, an ondansetron-containing or palonosetron-containing formulation can include one or more excipients, such as dyes, flavors, binders, emollients, fillers, lubricants and preservatives. In another embodiment, a serotonin receptor antagonist formulation can include one or more of cornstarch, lactose, talc, magnesium stearate, sucrose, gelatin, calcium stearate, silicon dioxide, shellac and glaze. In another embodiment, an ondansetron-containing formulation or palonosetron-containing formulation can include one or more of cornstarch, lactose, talc, magnesium stearate, sucrose, gelatin, calcium stearate, silicon dioxide, shellac, glaze and microcrystalline cellulose. In another embodiment, a serotonin receptor antagonist formulation can include one or more of lactose, microcrystalline cellulose, pregelatinized starch, hypromellose, magnesium stearate, titanium dioxide, triacetin, and iron oxide yellow. In another embodiment, an ondansetron formulation or palonosetron formulation can include one or more of lactose, microcrystalline cellulose, pregelatinized starch, hypromellose, magnesium stearate, titanium dioxide, triacetin, and iron oxide yellow. In another embodiment, a serotonin receptor antagonist formulation can include one or more of aspartame, gelatin, mannitol, methylparaben sodium and propylparaben sodium. In another embodiment, a serotonin receptor antagonist formulation can include one or more of citric acid anhydrous, purified water, sodium benzoate, sodium citrate and sorbitol. In another embodiment, an ondansetron formulation or palonosetron formulation can include one or more of aspartame, gelatin, mannitol, methylparaben sodium and propylparaben sodium. In another embodiment, an ondansetron formulation or palonosetron formulation can include one or more of citric acid anhydrous, purified water, sodium benzoate, sodium citrate and sorbitol.

In an embodiment, a serotonin receptor antagonist, ondansetron-containing or palonosetron-containing formulation can include one or more excipients selected from lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup and methyl cellulose. A serotonin receptor antagonist, ondansetron-containing or palonosetron-containing formulation can also include one or more of lubricating agents (such as talc); magnesium stearate; mineral oil; wetting agents; emulsifying and suspending agents; preserving agents, such as methyl and propylhydroxy-benzoates; sweetening agents; and flavoring agents. In another embodiment, a serotonin receptor antagonist formulation can include one or more of mannitol, edentate disodium dihydrate, trisodium citrate dihydrate and citric acid monohydrate. In another embodiment, a serotonin receptor antagonist formulation can include one or more of citric acid anhydrous, hypromellose, lactose, magnesium stearate, cellulose (or microcrystalline cellulose), polyethylene glycol, polysorbate, sodium starch glycolate and titanium dioxide. In another embodiment, a serotonin receptor antagonist formulation can include one or more of mannitol, phenol, acetate and water. In another embodiment, an ondansetron formulation or palonosetron formulation can include one or more of mannitol, edentate disodium dihydrate, trisodium citrate dihydrate and citric acid monohydrate. In another embodiment, an ondansetron formulation or palonosetron formulation can include one or more of citric acid anhydrous, hypromellose, lactose, magnesium stearate, cellulose (or microcrystalline cellulose), polyethylene glycol, polysorbate, sodium starch glycolate and titanium dioxide. In another embodiment, an ondansetron formulation or palonosetron formulation can include one or more of mannitol, phenol, acetate and water.

In an embodiment, a serotonin receptor antagonist formulation comprising ondansetron includes one or more of the inactive ingredients (excipients) citric acid anhydrous, sodium benzoate, sodium citrate, sorbitol and water. In another embodiment, a serotonin receptor antagonist formulation comprising palonosetron includes one or more of the inactive ingredients mannitol, disodium edetate, citrate and water.

In an embodiment, a serotonin receptor antagonist formulation having little or no preservatives is provided. In another embodiment, an ondansetron-containing formulation having little or no preservatives is provided. In another embodiment, a palonosetron-containing formulation having little or no preservatives is provided.

Serotonin receptor antagonist formulations of embodiments of the invention, including methods and processes for forming such formulations, can be combined or modified with other serotonin receptor antagonist formulations and methods for forming serotonin receptor antagonist formulations, such as, for example, compounds, formulations and/or methods provided by U.S. Pat. Nos. 5,478,949 to Bod et al. (“Process for preparing ondansetron”), 5,344,658 to Collin (“Process and composition using ondansetron”) and 5,854,270 to Gambhir (“Oral compositions containing ondansetron”) and U.S. Pat. No. 7,737,280 to Rossetto et al. (“Processes for preparing palonosetron salts”), and U.S. Patent Publication Nos. 2004/0198794 to Westheim et al. (“Ondansetron forms and processes of making the same”) and 2006/0069114 to Calderari et al. (“Liquid pharmaceutical formulations of palonosetron”), which are entirely incorporated herein by reference.

### Injection and Microinjection Systems

In another aspect of the invention, injection systems are provided for the delivery serotonin receptor antagonist compounds of embodiments of the invention. In embodiments, injection systems include microinjection systems. Microinjection systems of embodiments of the invention can be configured for subcutaneous, transdermal or intradermal drug delivery. Microinjection systems of embodiments of the invention can provide for improved delivery efficiency and absorption times in relation to traditional syringes. Microinjection systems of embodiments of the invention can include one or more microneedles configured to deliver serotonin receptor antagonist formulations or drug formulations, such as, for example, a formulation comprising ondansetron or palonosetron.

In another embodiment, a microinjection system can include a solid microneedle system having one or more solid microneedles, wherein at least a portion of the one or more solid microneedles are coated with a serotonin receptor antagonist drug formulation, such as ondansetron or palonosetron. In another embodiment, a microinjection system can include a hollow microneedle system having one or more hollow microneedles. The one or more hollow microneedles can include fluid passages for directing a formulation having a serotonin receptor antagonist drug formulation from a reservoir to a subject.

In some embodiments, solid microneedle systems are provided having one or more microneedles (or microneedle assemblies). In an embodiment, the solid microneedle systems can be configured for the delivery of serotonin receptor antagonist drug formulations, up to and including about 0.01 mg, or 0.02 mg, or 0.03 mg, or 0.04 mg, or 0.05 mg, or 0.06 mg, or 0.07 mg, or 0.08 mg, or 0.09 mg, or 0.1 mg, or 0.2 mg, or 0.3 mg, or 0.4 mg, or 0.5 mg, or 0.6 mg, or 0.7 mg, or 0.8 mg, or 0.9 mg, or 1 mg, or 2 mg, or 3 mg, or 4 mg, or 5 mg, or 6 mg, or 7 mg, or 8 mg, or 9 mg, or 10 mg, or 11 mg, or 12 mg, or 13 mg, or 14 mg, or 15 mg, or 16 mg, or 17 mg, or 18 mg, or 19 mg, or mg, or 21 mg, or 22 mg, or 23 mg, or 24 mg, or 25 mg, or 26 mg, or 27 mg, or 28 mg, or 29 mg, or 30 mg, or 31 mg, or 32 mg, or 33 mg, or 34 mg, or 35 mg, or 36 mg, or 37 mg, or 38 mg, or 39 mg, or 40 mg of a serotonin receptor antagonist drug formulation. In another embodiment, the solid microneedle systems can be configured for the delivery of ondansetron drug formulations, up to and including about 0.01 mg, or 0.02 mg, or 0.03 mg, or 0.04 mg, or 0.05 mg, or 0.06 mg, or 0.07 mg, or 0.08 mg, or 0.09 mg, or 0.1 mg, or 0.2 mg, or 0.3 mg, or 0.4 mg, or 0.5 mg, or 0.6 mg, or 0.7 mg, or 0.8 mg, or 0.9 mg, or 1 mg, or 2 mg, or 3 mg, or 4 mg, or 5 mg, or 6 mg, or 7 mg, or 8 mg, or 9 mg, or 10 mg, or 11 mg, or 12 mg, or 13 mg, or 14 mg, or 15 mg, or 16 mg, or 17 mg, or 18 mg, or 19 mg, or 20 mg, or 21 mg, or 22 mg, or 23 mg, or 24 mg, or 25 mg, or 26 mg, or 27 mg, or 28 mg, or 29 mg, or 30 mg, or 31 mg, or 32 mg, or 33 mg, or 34 mg, or 35 mg, or 36 mg, or 37 mg, or 38 mg, or 39 mg, or 40 mg of an ondansetron drug formulation. In another embodiment, the solid microneedle systems can be configured for the delivery of palonosetron drug formulations, up to and including about 0.01 mg, or 0.02 mg, or 0.03 mg, or 0.04 mg, or 0.05 mg, or 0.06 mg, or 0.07 mg, or 0.08 mg, or 0.09 mg, or 0.1 mg, or 0.2 mg, or 0.3 mg, or 0.4 mg, or 0.5 mg, or 0.6 mg, or 0.7 mg, or 0.8 mg, or 0.9 mg, or 1 mg, or 2 mg, or 3 mg, or 4 mg, or 5 mg, or 6 mg, or 7 mg, or 8 mg, or 9 mg, or 10 mg, or 11 mg, or 12 mg, or 13 mg, or 14 mg, or 15 mg, or 16 mg, or 17 mg, or 18 mg, or 19 mg, or 20 mg, or 21 mg, or 22 mg, or 23 mg, or 24 mg, or 25 mg, or 26 mg, or 27 mg, or 28 mg, or 29 mg, or 30 mg, or 31 mg, or 32 mg, or 33 mg, or 34 mg, or 35 mg, or 36 mg, or 37 mg, or 38 mg, or 39 mg, or 40 mg of a palonosetron drug formulation.

In embodiments, microinjection devices and systems are provided. In an embodiment, a microinjection system is provided configured to deliver at least 0.001 mg, or 0.01 mg, or 0.1 mg, or 1 mg, or 10 mg, or 20 mg, or 30 mg, or 40 mg of a serotonin receptor antagonist formulation in 1 mL of the serotonin receptor antagonist formulation. In another embodiment, a microinjection system is provided configured to deliver at least 0.1 mg, or 1 mg, or 10 mg, or 20 mg, or 30 mg, or 40 mg of an ondansetron formulation in 1 mL of the ondansetron formulation. In another embodiment, a microinjection system is provided configured to deliver at least 0.001 mg, or 0.01 mg, or 0.1 mg, or 1 mg of a palonosetron formulation in 1 mL of the palonosetron formulation.

In an embodiment, solid microneedle systems can include between about 300 and 1500 solid microneedles. Each microneedle can have a height between about 250 and 700 μm tall. In another embodiment, each microneedle can be coated with a serotonin receptor antagonist-containing drug or vaccine, such as a drug formulation comprising ondansetron or palonosetron. In an embodiment, the tip of each microneedle can be coated with a serotonin receptor antagonist drug formulation. A solid microneedle system can be integrated into a user-wearable device. Upon application, the microneedles penetrate stratum corneum for delivery of the serotonin receptor antagonist drug formulation. The microneedles can remain in the skin for a desirable or predetermined period of time, such as a length of time selected to permit the delivery of the serotonin receptor antagonist-containing drug to a subject. Such time can be between about 30 seconds and 60 minutes. Serotonin receptor antagonist drug formulations can be kept in a dry state, which can enhance stability, allowing for room temperature storage of the formulations. Solid microneedle system can be configured for single or multiple uses.

In an embodiment, a microinjection device having one or more hollow microneedles is provided. The one or more hollow microneedles can be configured to deliver serotonin receptor antagonists of embodiments of the invention. In another embodiment, microinjection devices can include a plurality of hollow microneedles. In another embodiment, a hollow microneedle system can be configured for the delivery of a serotonin receptor antagonist drug formulation in liquid form, from about 0.01 mL up to and including about 3 mL of a serotonin receptor antagonist drug formulation, such as about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.0, 1.00, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.60, 1.61, 1.62, 1.63, 1.64, 1.65, 1.66, 1.67, 1.68, 1.69, 1.70, 1.71, 1.72, 1.73, 1.74, 1.75, 1.76, 1.77, 1.78, 1.79, 1.80, 1.81, 1.82, 1.83, 1.84, 1.85, 1.86, 1.87, 1.88, 1.89, 1.90, 1.91, 1.92, 1.93, 1.94, 1.95, 1.96, 1.97, 1.98, 1.99, 2.00, 2.01, 2.02, 2.03, 2.04, 2.05, 2.06, 2.07, 2.08, 2.09, 2.10, 2.11, 2.12, 2.13, 2.14, 2.15, 2.16, 2.17, 2.18, 2.19, 2.20, 2.21, 2.22, 2.23, 2.24, 2.25, 2.26, 2.27, 2.28, 2.29, 2.30, 2.31, 2.32, 2.33, 2.34, 2.35, 2.36, 2.37, 2.38, 2.39, 2.40, 2.41, 2.42, 2.43, 2.44, 2.45, 2.46, 2.47, 2.48, 2.49, 2.50, 2.51, 2.52, 2.53, 2.54, 2.55, 2.56, 2.57, 2.58, 2.59, 2.60, 2.61, 2.62, 2.63, 2.64, 2.65, 2.66, 2.67, 2.68, 2.69, 2.70, 2.71, 2.72, 2.73, 2.74, 2.75, 2.76, 2.77, 2.78, 2.79, 2.80, 2.81, 2.82, 2.83, 2.84, 2.85, 2.86, 2.87, 2.88, 2.89, 2.90, 2.91, 2.92, 2.93, 2.94, 2.95, 2.96, 2.97, 2.98, 2.99, or 3.00 mL. In another embodiment, a hollow microneedle system can be configured for the delivery of a serotonin receptor antagonist drug formulation in liquid form, from about 0.01 ml to 6 mL, or 0.01 mL to 3 mL, or 0.02 mL to 2 mL of a serotonin receptor antagonist drug formulation. In another embodiment, hollow microneedle systems can include about 18 hollow microneedles per cm². Each microneedle can have a height of about 900 μm. A hollow microneedle system can be integrated into user-wearable device. Upon application, the microneedles penetrate the skin. Small channels in each microneedle allow for the flow of a fluid having a serotonin receptor antagonist drug formulation from the device into the skin. In another embodiment, the delivery time can be between about 0.1 seconds and 2 hours, or between about 10 seconds and 1 hour, or between about 30 seconds and 40 minutes, or between about 1 minute and 30 minutes. The infusion time can be dependent on the viscosity and volume of the serotonin receptor antagonist-containing fluid.

In an embodiment, a microinjection device having one or more hollow microneedles is provided. The one or more hollow microneedles can be configured to deliver ondansetron. In another embodiment, microinjection devices can include a plurality of hollow microneedles. In another embodiment, a hollow microneedle system can be configured for the delivery of an ondansetron drug formulation in liquid form, from about 0.01 mL up to and including about 3 mL of an ondansetron drug formulation, such as about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.0, 1.00, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.60, 1.61, 1.62, 1.63, 1.64, 1.65, 1.66, 1.67, 1.68, 1.69, 1.70, 1.71, 1.72, 1.73, 1.74, 1.75, 1.76, 1.77, 1.78, 1.79, 1.80, 1.81, 1.82, 1.83, 1.84, 1.85, 1.86, 1.87, 1.88, 1.89, 1.90, 1.91, 1.92, 1.93, 1.94, 1.95, 1.96, 1.97, 1.98, 1.99, 2.00, 2.01, 2.02, 2.03, 2.04, 2.05, 2.06, 2.07, 2.08, 2.09, 2.10, 2.11, 2.12, 2.13, 2.14, 2.15, 2.16, 2.17, 2.18, 2.19, 2.20, 2.21, 2.22, 2.23, 2.24, 2.25, 2.26, 2.27, 2.28, 2.29, 2.30, 2.31, 2.32, 2.33, 2.34, 2.35, 2.36, 2.37, 2.38, 2.39, 2.40, 2.41, 2.42, 2.43, 2.44, 2.45, 2.46, 2.47, 2.48, 2.49, 2.50, 2.51, 2.52, 2.53, 2.54, 2.55, 2.56, 2.57, 2.58, 2.59, 2.60, 2.61, 2.62, 2.63, 2.64, 2.65, 2.66, 2.67, 2.68, 2.69, 2.70, 2.71, 2.72, 2.73, 2.74, 2.75, 2.76, 2.77, 2.78, 2.79, 2.80, 2.81, 2.82, 2.83, 2.84, 2.85, 2.86, 2.87, 2.88, 2.89, 2.90, 2.91, 2.92, 2.93, 2.94, 2.95, 2.96, 2.97, 2.98, 2.99, or 3.00 mL. In another embodiment, a hollow microneedle system can be configured for the delivery of an ondansetron-containing drug formulation in liquid form, from about 0.01 ml to 6 mL, or 0.01 mL to 3 mL, or 0.02 mL to 2 mL of an ondansetron-containing drug formulation. In another embodiment, hollow microneedle systems can include about 18 hollow microneedles per cm². Each microneedle can have a height of about 900 μm. A hollow microneedle system can be integrated into user-wearable device. Upon application, the microneedles penetrate the skin. Small channels in each microneedle allow for the flow of a fluid having an ondansetron-containing drug formulation from the device into the skin. In another embodiment, the delivery time can be between about 0.1 seconds and 2 hours, or between about 10 seconds and 1 hour, or between about 30 seconds and 40 minutes, or between about 1 minute and 30 minutes. The infusion time can be dependent on the viscosity and volume of the ondansetron-containing fluid.

In an embodiment, a microinjection device having one or more hollow microneedles is provided. The one or more hollow microneedles can be configured to deliver palonosetron. In another embodiment, microinjection devices can include a plurality of hollow microneedles. In another embodiment, a hollow microneedle system can be configured for the delivery of a palonosetron drug formulation in liquid form, from about 0.01 mL up to and including about 3 mL of a palonosetron drug formulation, such as about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.0, 1.00, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.60, 1.61, 1.62, 1.63, 1.64, 1.65, 1.66, 1.67, 1.68, 1.69, 1.70, 1.71, 1.72, 1.73, 1.74, 1.75, 1.76, 1.77, 1.78, 1.79, 1.80, 1.81, 1.82, 1.83, 1.84, 1.85, 1.86, 1.87, 1.88, 1.89, 1.90, 1.91, 1.92, 1.93, 1.94, 1.95, 1.96, 1.97, 1.98, 1.99, 2.00, 2.01, 2.02, 2.03, 2.04, 2.05, 2.06, 2.07, 2.08, 2.09, 2.10, 2.11, 2.12, 2.13, 2.14, 2.15, 2.16, 2.17, 2.18, 2.19, 2.20, 2.21, 2.22, 2.23, 2.24, 2.25, 2.26, 2.27, 2.28, 2.29, 2.30, 2.31, 2.32, 2.33, 2.34, 2.35, 2.36, 2.37, 2.38, 2.39, 2.40, 2.41, 2.42, 2.43, 2.44, 2.45, 2.46, 2.47, 2.48, 2.49, 2.50, 2.51, 2.52, 2.53, 2.54, 2.55, 2.56, 2.57, 2.58, 2.59, 2.60, 2.61, 2.62, 2.63, 2.64, 2.65, 2.66, 2.67, 2.68, 2.69, 2.70, 2.71, 2.72, 2.73, 2.74, 2.75, 2.76, 2.77, 2.78, 2.79, 2.80, 2.81, 2.82, 2.83, 2.84, 2.85, 2.86, 2.87, 2.88, 2.89, 2.90, 2.91, 2.92, 2.93, 2.94, 2.95, 2.96, 2.97, 2.98, 2.99, or 3.00 mL. In another embodiment, a hollow microneedle system can be configured for the delivery of a palonosetron-containing drug formulation in liquid form, from about 0.01 ml to 6 mL, or 0.01 mL to 3 mL, or 0.02 mL to 2 mL of a palonosetron-containing drug formulation. In another embodiment, hollow microneedle systems can include about 18 hollow microneedles per cm². Each microneedle can have a height of about 900 μm. A hollow microneedle system can be integrated into user-wearable device. Upon application, the microneedles penetrate the skin. Small channels in each microneedle allow for the flow of a fluid having a palonosetron-containing drug formulation from the device into the skin. In another embodiment, the delivery time can be between about 0.1 seconds and 2 hours, or between about 10 seconds and 1 hour, or between about 30 seconds and 40 minutes, or between about 1 minute and 30 minutes. The infusion time can be dependent on the viscosity and volume of the palonosetron-containing fluid.

In an embodiment, a microinjection device comprises a plurality of hollow microneedles configured to deliver a serotonin receptor antagonist formulation to a subject. In an embodiment, each microneedle is formed of a polymeric material. In another embodiment, each microneedle is formed of a metallic material, such as an elemental metal or a metal alloy. In yet another embodiment, each microneedle is formed of a semiconductor material. In still another embodiment, each microneedle is formed of an insulating material. In still another embodiment, each microneedle is formed of one or more of a metallic material, a semiconductor material and an insulating material.

In an embodiment, a system for subcutaneous, transdermal or intradermal delivery of a serotonin receptor antagonist to a subject comprises a serotonin receptor antagonist formulation; a microneedle array for delivering the serotonin receptor antagonist formulation to a subject; and one or more chambers in fluid communication with the microneedle array, the one or more chambers configured to hold the serotonin receptor antagonist formulation. The system can be configured to deliver to a subject a formulation comprising ondansetron or derivatives of ondansetron, or palonosetron or derivatives of palonosetron.

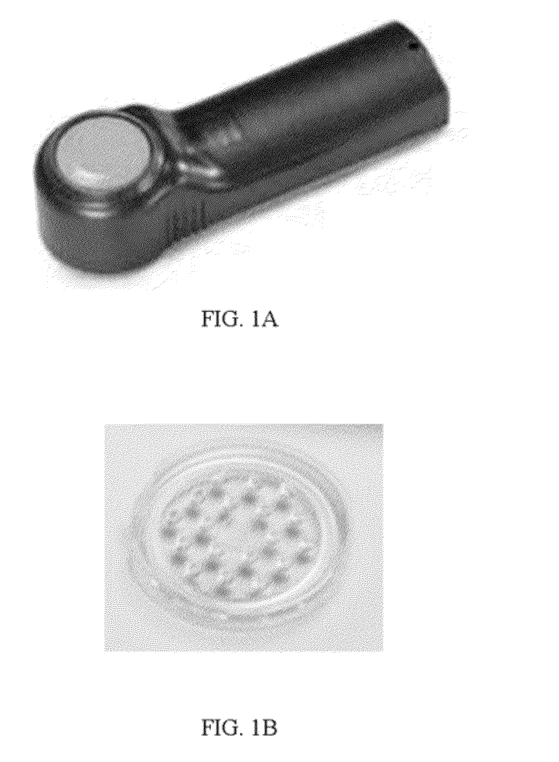

FIG. 1A illustrates a microinjection device configured to deliver serotonin receptor antagonists, in accordance with an embodiment of the invention. The microinjection device includes a handle portion configured to permit a user to hold or grip the microinjection device. FIG. 1B illustrates an array of microneedles mountable to the microinjection device of FIG. 1A, in accordance with an embodiment of the invention. Upon application, the microneedles can penetrate the skin of a subject with minimal discomfort. Small channels in each microneedle can allow for fluid flow from the device into the subject's skin.

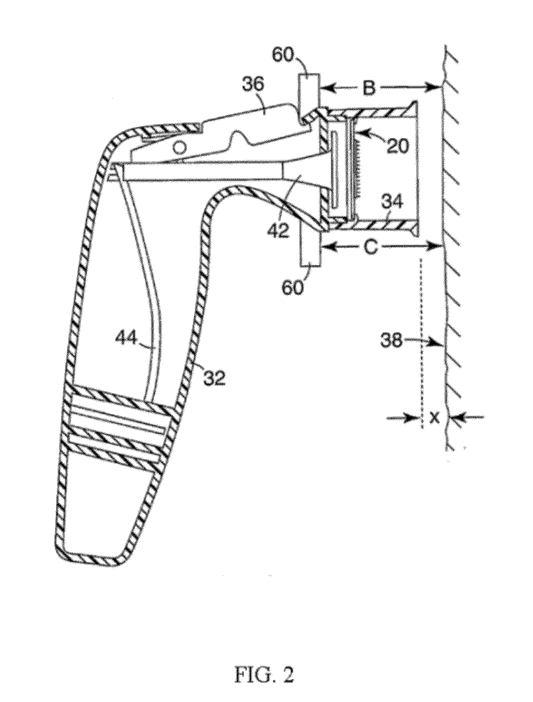

FIG. 2 illustrates a microinjection device having an array of microneedles (also “microneedle array application device” herein), in accordance with an embodiment of the invention. The application device includes a patch 20, microneedle array 22, collar 34, actuator 36, piston 42, driver 44, holding tabs 50 and distance sensors 60. In the illustrated embodiment, the actuator 36 of the device has not been engaged. The driver 44 has stored energy and the piston 42 is not in contact with the patch 20, which is retained within the collar 34 of the application device. The application device has distance sensors 60 that sense distances “B” and “C” between the sensor and a skin surface 38. A user can bring the applicator in proximity to the skin surface 38 so that the distances “B” and “C” 40 can be adjusted so that a distance, “x”, between the end of the collar 34 and the skin surface 38 can be as desired. Once the distance and orientation of the application device is as desired (e.g., when “B” and “C” are equivalent and the distance “x” is less than the distance that the piston 42 protrudes from the device after activation), then the application device can be triggered.

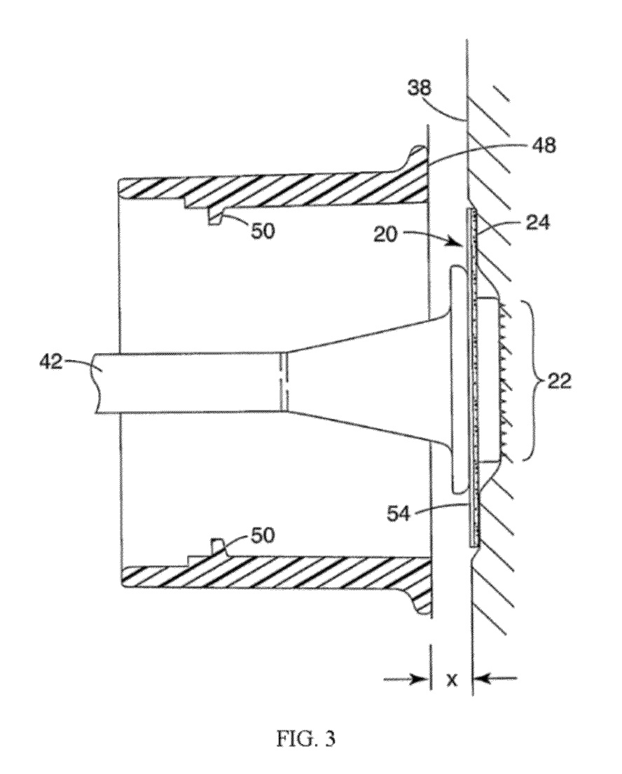

FIG. 3 illustrates a portion of the application device of FIG. 2, in accordance with an embodiment of the invention. The application device is in the second released or triggered position, where the actuator 36 has been engaged, allowing the driver 44 to move the piston 42 towards the patch 20, thereby removing the patch from the holding tabs 50, propelling the patch 20 beyond an open distal end 48 of the collar 34 and pressing the microneedle array 22 and a skin facing adhesive 24 against the skin 38. The piston 42 can then be removed from contact with the patch 20, thereby leaving the patch 20 in place on the skin 38. In an alternative embodiment, the piston 42 can propel the patch 20 and array 22 from the application device and the patch 20 and array 22 can travel part of the distance in air (not shown) before impacting with the skin surface 38.

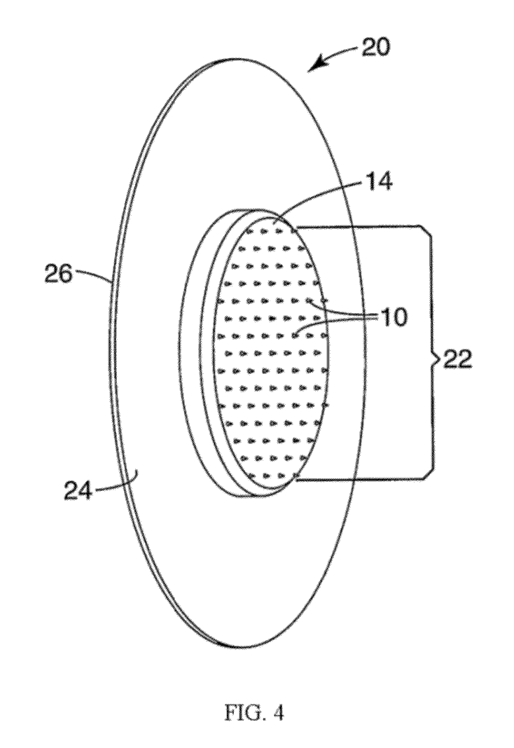

FIG. 4 illustrates a microneedle device comprising a patch 20 in the form of a combination of an array 22, pressure sensitive adhesive 24 and backing 26, in accordance with an embodiment of the invention. A portion of the array 22 is illustrated with microneedles 10 protruding from a microneedle substrate surface 14. The microneedles 10 can be arranged in any desired pattern or distributed over the microneedle substrate surface 14 randomly. In an embodiment, the microneedles 10 are configured for delivering a serotonin receptor antagonist-containing formulation, such as an ondansetron-containing formulation or palonosetron-containing formulation, to a subject. As shown, the microneedles 10 are arranged in uniformly spaced rows. In an embodiment, arrays of the present invention have a skin-facing surface area of more than about 0.1 cm² and less than about 20 cm², or more than about 0.5 cm² and less than about 5 cm². As shown, a portion of the substrate surface 16 of the patch 20 is non-patterned. In an embodiment the non-patterned surface has an area of more than about 1 percent and less than about 75 percent of the total area of the device surface that faces a skin surface of a subject. In an embodiment, the non-patterned surface can have an area of more than about 0.10 square inch (0.65 cm²) to less than about 1 square inch (6.5 cm²). In another embodiment (not shown), the microneedles can be disposed over substantially the entire surface area of the array 22.

The microneedle devices useful in various embodiments of the invention can comprise any of a variety of configurations, such as the structures and configuration disclosed in U.S. Patent Publication No. 2003/0045837 to Delmore et al., U.S. Patent Publication No. 2005/0261631 to Graham et al., U.S. Pat. No. 6,091,975 to Daddona et al., U.S. Pat. No. 6,312,612 to Sherman et al., U.S. Pat. No. 6,379,324 to Garstein et al., and WO/2000/74766 to Garstein et al., which are entirely incorporated herein by reference.

In an embodiment, a microinjection device is provided having an array of microneedles, wherein the microneedles in the array include tapered structures that include at least one channel formed in the outside surface of each microneedle. The microneedles can include bases that are elongated in one direction. The channels in microneedles with elongated bases can extend from one of the ends of the elongated bases towards the tips of the microneedles. The channels formed along the sides of the microneedles can optionally be terminated short of the tips of the microneedles. The microneedle arrays can also include conduit structures formed on the surface of the substrate on which the microneedle array is located. The channels in the microneedles can be in fluid communication with the conduit structures. In another embodiment, each of the microneedles can include a truncated tapered shape and a controlled aspect ratio. In yet another embodiment, microneedles can include blade-like microprotrusions for piercing the skin. In still another embodiment, each of the microneedles can include a hollow central channel. In still another embodiment, each of the microneedles can be hollow and include at least one longitudinal blade at the top surface of tip of a microneedle.

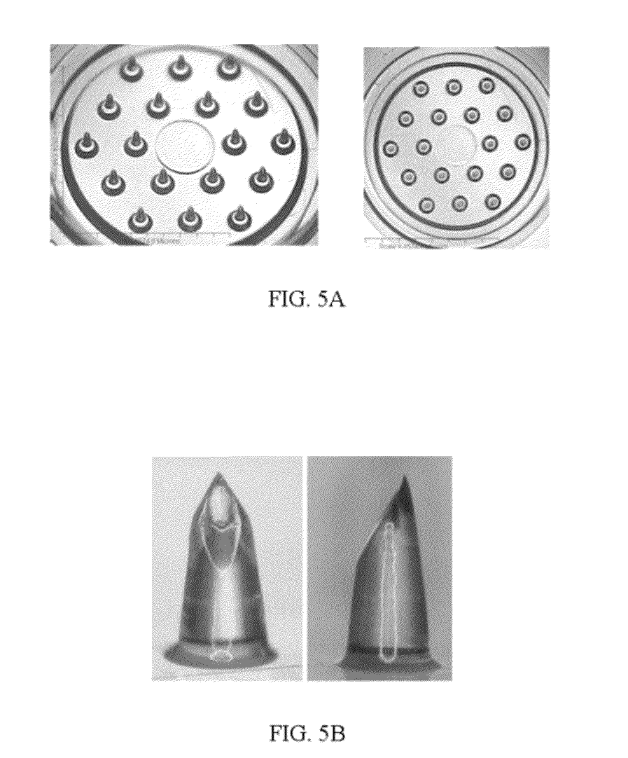

With reference to FIG. 5A, an array of microneedles is shown, in accordance with an embodiment of the invention. FIG. 5B shows a cross-section of a microneedle in the array, in accordance with an embodiment of the invention. The microneedles can be formed of a polymeric material, such as a medical-grade polymeric material. They can be configured to overcome the barrier properties of the stratum corneum to deliver to a subject serotonin receptor antagonists of embodiments of the invention, such as ondansetron or palonosetron. In an embodiment, microneedles can be modeled as mini hypodermic needles, each having a height between about 100 μm and 1000 μm, or between about 300 μm and 950 μm, or between about 500 μm and 900 μm. In an embodiment, the array can include 18 microneedles in an array area of about 1 cm². Each microneedle can include a fluid passage for delivering serotonin receptor antagonists, each fluid passage running the length of a microneedle. Each microneedle can include a tip portion configured to pierce a subject's skin.

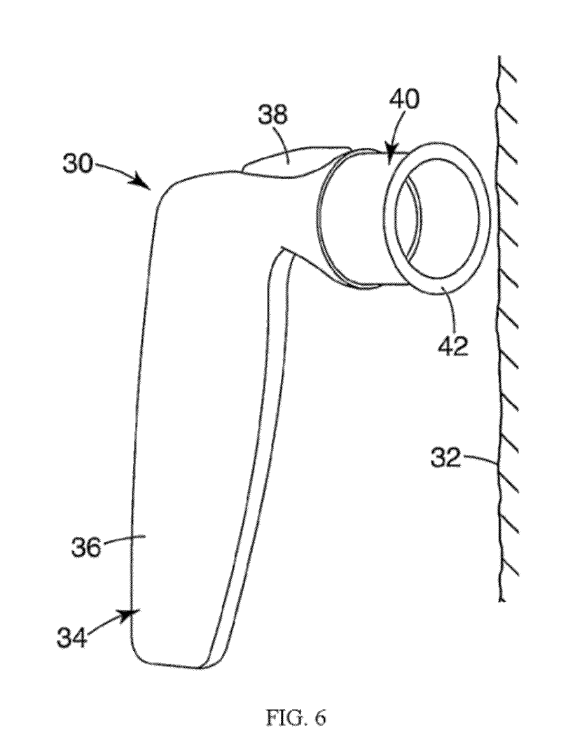

FIG. 6 illustrates a microneedle application device 30 and a skin surface 32, in accordance with an embodiment of the invention. The microneedle device 30 can be used to deliver serotonin receptor antagonist compounds of embodiments of the invention, such as ondansetron or palonosetron. In an embodiment, the microneedle device 30 can be used to deliver an ondansetron-containing formulation or a palonosetron-containing formulation. In another embodiment, the microneedle device 30 can be used to deliver an ondansetron-containing formulation or a palonosetron-containing formulation, which can include other active or inactive ingredients (see above).

With continued reference to FIG. 6, the microneedle application device 30 can be used to deploy patches that include a microneedle array to a surface, such as to the skin surface 32. The device 30 includes a housing 34 with a gripping portion 36, a trigger 38 and a collar 40. The collar 40 defines an outward-facing contact portion 42. In an embodiment, the collar 40 is detachable from the housing 34, and can be disposable or reusable. As shown in FIG. 6, the collar 40 is a unitary member of generally cylindrical shape, and contact portion 42 is generally annular in shape. In further embodiments, the collar 40 can have nearly any shape and configuration. For example, the collar 40 can have a rectangular, triangular, oval, or other shape or combination of shapes. The contact portion 42 will typically have a shape corresponding to the shape of the collar 40. In addition, the collar 40 need not be unitary, and can be configured to form a number of discrete feet or supports that collectively define the contact portion 42.

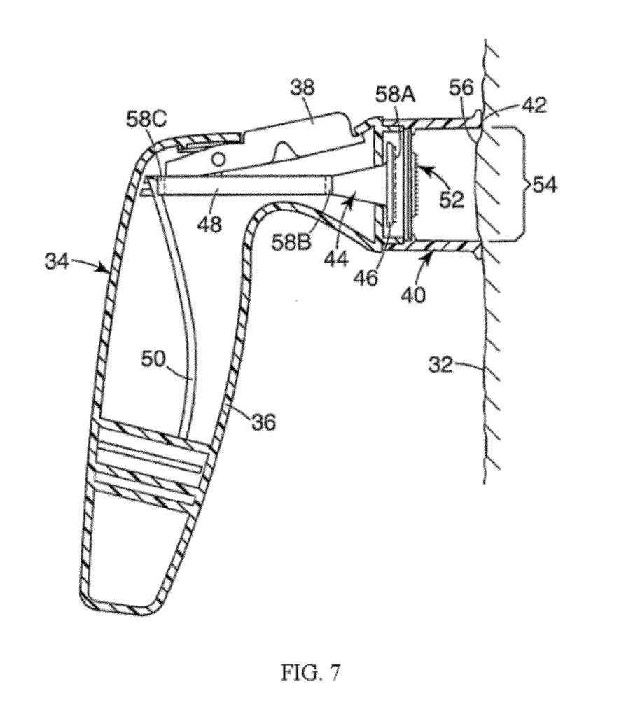

FIG. 7 is a cross sectional side view of the microneedle application device 30 of FIG. 6, in accordance with an embodiment of the invention. The device 30 includes a microneedle array patch 52; the device 30 is positioned against the skin surface 32. The device 30 includes a support member or actuator. In the illustrated embodiment of FIG. 7, the support member or actuator can be a piston 44 having a pad 46 and a shaft 48. In alternative embodiments, any type of mechanical, electromechanical, pneumatic, or other type of support member or actuator can be used.

With continued reference to FIG. 7, a driver 50 capable of storing energy engages the shaft 48 of the piston 44, and can accelerate the piston 44 to a desired velocity. For example, the driver 50 can be in the form of a mechanical spring (e.g., a coil spring, leaf spring, etc.), compressed resilient member (e.g., rubber, etc.), compressed fluids (e.g., air, liquids, etc.), piezoelectric structure, electromagnetic structure, etc. The collar 40 can hold a patch 52, carrying a microneedle array, prior to patch application.

With continued reference to FIG. 7, during operation, the microneedle application device 30 can be positioned with the collar 40 near a desired application site. The contact portion 42 of the collar 40 is placed in contact with the skin surface 32, and the contact portion 42 defines a target patch application site 54 on the skin surface 32. A user can apply force to the microneedle application device 30 at the gripping portion 36 of the housing 34. At least a portion of that force can be transmitted through the collar 40 to the skin 32. That force can be referred to as a “pushdown force”. A “dome” 56 is generally created at the target site 54, as the skin 32 responds to the pushdown force. This “dome” has parameters of height and firmness. Both of these parameters of the dome can be dependent upon the force applied to the applicator during microneedle application device 30 positioning. The depth of penetration of a microneedle array is related to the application site, i.e., soft and fatty areas of a body versus firm muscular areas of the body. Skin characteristics can vary from one individual to another, and particular characteristics of skin can vary across subjects (e.g., patients) and across selected application sites on individual subjects. Such variations can affect characteristics of the dome 56. In addition, a “pushback force” is exerted by the skin 32 in response to the pushdown force. The pushback force is generally directed in a direction directly opposed to the direction of the pushdown force, although specific relationships can be complex and will vary depending on the particular application site.

With continued reference to FIG. 7, a force sensor can be coupled to the piston 44 at either end or anywhere along the length of piston 44, for example, at location 58A, 58B and/or 58C (jointly referred to as sensor 58). The sensor 58 can be capable of sensing applied mechanical forces, such as pushback force at the piston 44. The sensor 58 can be a strain gauge, variable capacitance sensor, or variable resistance sensor. In an embodiment, the sensor 58 can comprise a variable resistance member having a semi-conducting polymer disposed between conductive layers or grids, where the resistance of the variable resistance member varies according to applied force. The variable resistance member can be further configured in a voltage divider, which converts the resistance of the member into a voltage signal output that can be measured to detect force applied to the sensor 58. An example of such a variable resistance member is disclosed in U.S. Pat. No. 5,209,967, which is herein incorporated by reference in its entirety. Other examples of aspects of such a variable resistance member are disclosed in U.S. Pat. Nos. 5,904,978 and 5,573,626, which are entirely incorporated herein by reference.

With continued reference to FIG. 7, in the microneedle application device 30, the piston 44 is moveable between a stored position and an extended position. In the stored position, energy is stored in the driver 50, and an actuator 38 secures the piston 44 in its stored position. The actuator 38 allows an operator to trigger the release of energy stored in the driver 50 to accelerate the piston 44 through the collar 40 and toward the patch 52.

In an embodiment, the microneedle application device 30 can be used to deliver the microneedle array patch 52 to the skin surface 32, in order to pierce the stratum corneum at the target application site 54 on a subject's skin. In an embodiment, the patch application device can be used to deliver a serotonin receptor antagonist formulation through the skin in a variation on transdermal delivery, or to the skin for intradermal or topical treatment, such as, e.g., vaccination. In another embodiment, the patch application device can be used to deliver an ondansetron formulation or a palonosetron formulation through the skin in a variation on transdermal delivery, or to the skin for intradermal or topical treatment, such as, e.g., vaccination. Alternatively, the microneedle array patch 52 can be used to pierce the stratum corneum before or after a pharmacological agent is applied to the skin surface in a separate step, thus being used as a pre- or post-treatment step.

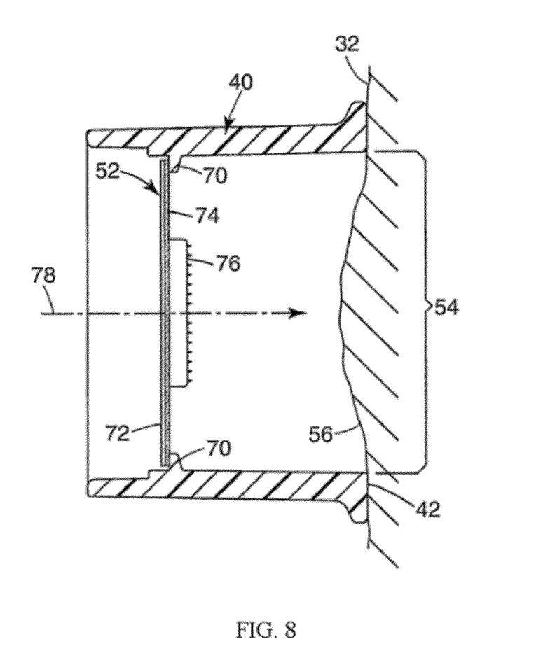

FIG. 8 shows an enlarged cross sectional view of the collar 40 of the microneedle application device 30 of FIGS. 6 and 7, positioned against the skin surface 32, in accordance with an embodiment of the invention. The collar 40 includes obstructions 70 on an interior portion thereof. The obstructions 70 can be configured to retain patches, such as the patch 52. Patch 52 can include a backing 72, an adhesive 74 (e.g., a pressure sensitive adhesive), and a microneedle array 76. A desired patch application path 78 is defined through the collar 40. The path 78 is substantially perpendicular to a plane in which the microneedle array 76 is retained by the obstructions 70 within the collar 40, and is generally perpendicular to the target application site 54. In an embodiment, it is desired that the patch 52 contact the target application site 54 with the patch 52 as close to parallel with the skin surface 32 as possible in order to promote proper microneedle array deployment and proper microneedle penetration of the stratum corneum.

With continued reference to FIG. 8, in operation, the patch 52 is moved along the patch application path 78. This patch movement can be accomplished by mechanically pushing the patch 52 with the piston 44. In alternative embodiments, the microneedle application device 30 can use other means for moving the patch 52. For example, the patch 52 can be moved pneumatically, without contacting a piston.

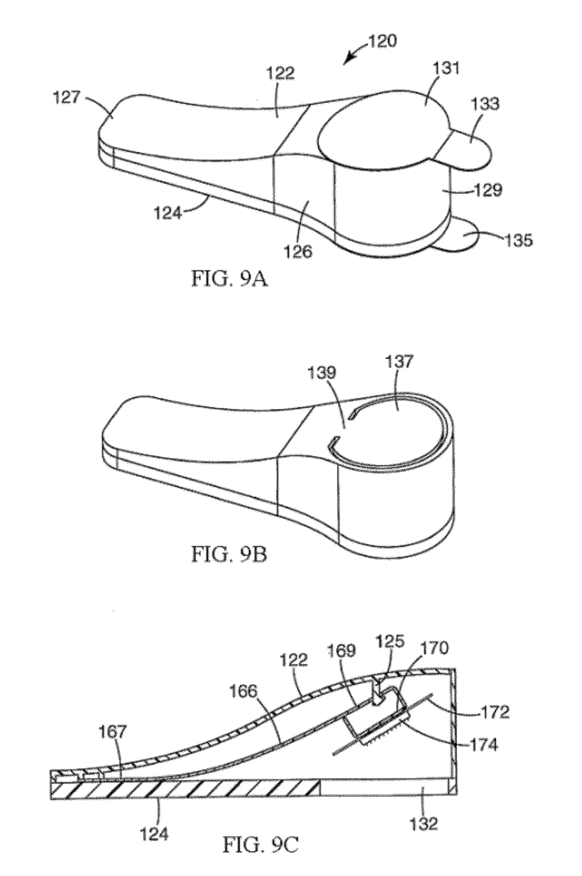

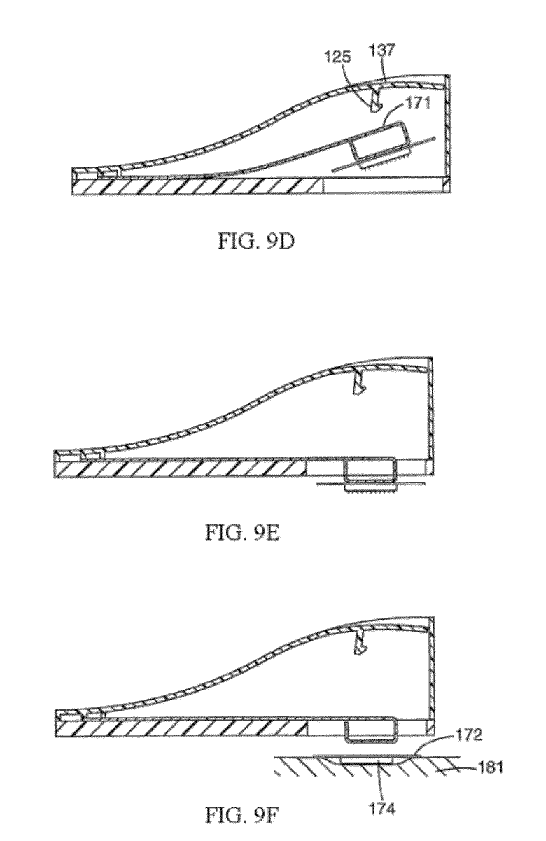

FIG. 9A is a perspective view of an applicator device 120 having a housing 122 that includes a base 124 and an upper cover structure 126, in accordance with an embodiment of the invention. The device is elongate in shape and has a first, tapered end 127 and a second end 129. The second end 129 has a top and bottom sealed by a top peelable seal 131 having a tab 133 and a bottom peelable seal 132 having a tab 135 (only tab 135 is visible in FIG. 9A). FIG. 9B shows the applicator device after the peelable seals 131, 132 have been removed. A trigger 137 is integrally formed in the top surface of the housing 122. The trigger is connected to the top surface of the housing at a single attachment point 139, thus allowing the trigger to be deflected downward by thumb or finger pressure (see FIG. 9D).

FIG. 9C is a cross-sectional view of the device of FIG. 9A, showing a patch 172 mounted on an impactor 170, in accordance with an embodiment of the invention. The impactor 170 is integrally formed with a drive member 166 having a length extending from a fixed end 167 attached to the housing 122 to a movable end 169. The drive member 166 is bendable along its length. A holding mechanism in the form of a latch uses a hook 125 attached to the housing 122. The hook 125 engages with a slot 171 in the movable end 169 of the drive member 166 to hold the movable end 169 of the drive member 166 away from the skin-contacting face 124 of the housing 122. The drive member can be any elongate, bendable member, such as, for example, a leaf spring. In use the device as shown in FIG. 9C is placed against a target surface, such as a skin surface (not shown). Depression of the trigger 137, as shown in FIG. 9D, causes the hook 125 to pivot, thus releasing the movable end 169 of the drive member 166 and allowing the drive member 166 to bias the patch 172 towards the skin-contacting face 124. FIG. 9E shows the drive member 166 fully deployed, having propelled the patch 172 past the skin-contacting face 124 so that the patch is pressed against the skin surface (not shown). FIG. 9F shows the device 120 being removed from the skin surface 181, leaving a patch 172 with a microneedle array 174 in place on the skin surface 181. As shown, the impactor 170 is shown as a curled end of a leaf spring, as this allows for a convenient means for providing a holding mechanism (via the slot 171 in the movable end 169 of the leaf spring) while also providing a separate patch contacting and holding surface. However, any variety of suitable shapes can be used for the movable end 169 of the drive member 166, including a flat leaf spring having no curled end.

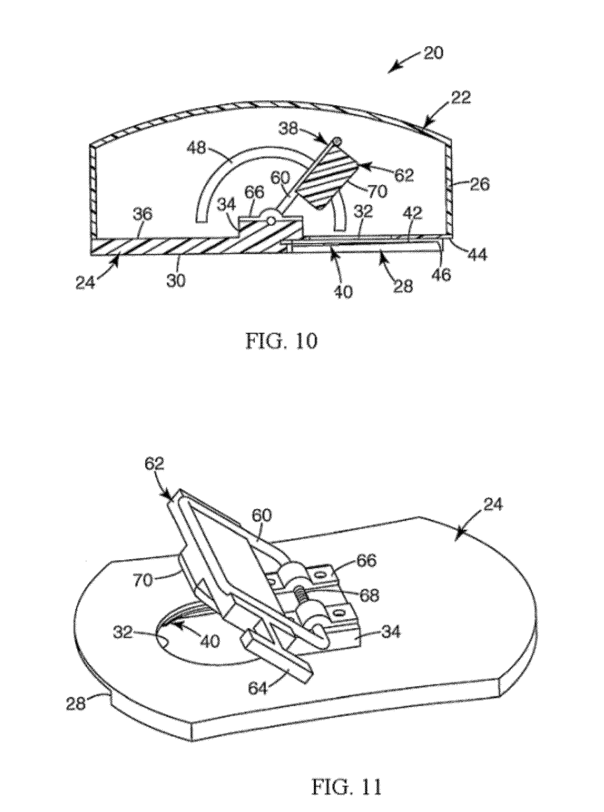

FIG. 10 illustrates an applicator device 20 having a housing 22 that includes a base 24 and an upper cover structure 26, in accordance with an embodiment of the invention. In an embodiment, the applicator device 20 is configured to deliver a serotonin receptor antagonist formulation to a subject. In an embodiment, the applicator device 20 is configured to deliver an ondansetron-containing formulation or a palonosetron-containing formulation to a subject. The base 24 can be rectangular in shape, and include a recess 28 located on a bottom face 30 thereof. A generally circular opening 32 is defined in the recess 28 of the base 24. A raised portion 34 is formed on an upper face 36 of the base 24 for holding a patch accelerating or patch applicator assembly 38. A mounting structure or retaining portion of the applicator device 20 is formed by a pair of retainers 40, also referred to as a first retainer and a second retainer, connected to the base 24 (only one retainer 40 is visible in FIG. 10). The retainer members 40 are generally elongate and each have a substantially flat upper surface 42 that is generally parallel to and facing a bottom portion 44 of the recess 28, and is spaced from the bottom face 30 (i.e., the skin-contacting face) of the base 24. The pair of retainer members 40 are located on opposite sides of the opening 32 and are connected to the base 24 at one side of the recess 28. The retainer members 40 define an opening 46 at one end for accepting patches between the retainer members 40 and the bottom portion 44 of the recess 28. The upper surfaces 42 of the retainer members 40 can be non-stick or release surfaces. A non-stick or release surface can be achieved, for example, by a non-stick or release coating applied to the upper surfaces 42. The non-stick or release coating can be selected according to the desired use of the applicator device 20. For instance, a release coating, such as a low surface energy silicone, fluoropolymer, or fluoro-silicone release coating, can be selected based upon the adhesives used with patches applied using the patch application device 20. In further embodiments, a blade or other cutting means can be provided as part of the mounting structure, for separating portions of items from patches mounted on the applicator.

With continued reference to FIG. 10, the upper cover structure 26 is connected to the base 24 at or near a perimeter of the base 24. The upper cover structure 26 is shaped to fit on the base 24, and defines a volume, which is selected to provide space for the patch accelerating assembly 38. In some embodiments, the housing 22 can also provide space for storing patches (e.g., a roll of patches) for eventual deployment by the applicator device 20. A slot 48 is defined in a side portion of the upper cover structure 26. In the illustrated embodiment of FIG. 10, the slot 48 is arcuate in shape and generally resembles a half circle, with the open portion of the half circle facing the base 24 of the housing 22. Both the base 24 and the upper cover structure 26 can be formed of a polymeric material.

FIG. 11 is a perspective view of a portion of the applicator device 20 of FIG. 10 with the upper cover portion 26 omitted to show interior portions of the device 20. As shown in FIG. 11, the patch acceleration assembly 38 includes a frame member 60, an impactor 62, a handle 64, a bracket 66, and a torsion spring 68. The torsion spring 68 serves as a drive member to bias the impactor relative to the housing. The bracket 66 is mounted to the raised portion 34 of the base 24 of the housing 22 and pivotally retains the frame member 60. In some instances the bracket 66 can be directly affixed to the base 24, for example, if the base has sufficient thickness to allow for placement of the torsion spring 68. The frame member 60 can be a wire formed as a rectangular loop. The impactor 62 is attached to the frame member 60 opposite the bracket 66, and is the portion of the patch acceleration assembly 38 that interfaces with a patch to move it (i.e., to accelerate it), that is, it is the patch contacting portion of the device. The impactor 62 has a patch contacting surface 70 that is configured according to characteristics of a desired application, for instance, based upon the shape of a patch to be applied. In the embodiment shown in FIG. 10, the patch contacting surface 70 is configured so that it is generally parallel to and aligned with the frame member 60. Furthermore, it will be generally aligned with the bottom face 30 of the device 20 when fully deployed. It other embodiments, the patch contacting surface 70 can be configured so that it is at another angle with respect to the frame member 60, and with respect to the bottom face 30 of the device 20 when fully deployed. Other such angles are possible. In an embodiment, the patch contacting surface 70 can be aligned so as to form an angle of between 4 and 15 degrees with the plane of the frame member. In an embodiment, the angle of the patch contacting surface 70 can be selected so that it is aligned with the back of the a patch resting on retaining members 40 when the patch contacting surface 70 contacts the patch. The impactor 62 can be formed of a polymer material. The handle 64 extends from the impactor 62, and can be integrally formed with the impactor 62. The handle 64 is arranged to protrude through the slot 48 in the upper cover structure 26 of the housing 22, allowing the impactor 62 position to be manipulated from outside the housing 22. It should be understood that FIG. 10 represents one configuration for manipulating the patch acceleration assembly 38. For example, a slot can be provided on the upper cover portion 26, thereby allowing the handle 64 or any other suitable actuation protrusion to protrude through the upper cover portion 26. Furthermore, the method for manipulating the patch acceleration assembly 38 need not be by means of a direct mechanical connection. For example, various linkages or gears can be provided such that a button or knob on the exterior of the housing 22 can be pressed or turned to manipulate the patch acceleration assembly 38. In a further example, the patch acceleration assembly 38 can be moved by a motor or solenoid that is electrically controlled by a button or knob on the exterior of the housing 22.

With continued reference to FIG. 11, the torsion spring 68 biases the frame 60 of the patch acceleration assembly 38 relative to the base 24 of the housing 22. The torsion spring 68 can be a conventional coiled spring steel torsion spring. By default, the torsion spring 68 biases the frame 60, and therefore also the impactor 62, toward the opening 32 in the base 24 of the housing 22. In a substantially de-energized state, the impactor is at rest and positioned near the opening 32 in the base 24 of the housing 22. By moving the handle 64 to position the impactor 62 away from the opening, along an arcuate path that can be defined by movement of the handle 64 along the slot 48 in the upper cover structure 26 of the housing 22, an operator can store potential energy in the torsion spring 68. Energy stored in the torsion spring 68 can be used to accelerate the impactor 62 toward a patch and also to accelerate a patch that has contacted the impactor 62. The amount of energy stored in the torsion spring 68 will vary depending on the amount of displacement of the impactor 62 away from the opening 32 and along the arcuate path. The appropriate torsion spring constant will depend upon a number of parameters, including the mass of the patch acceleration assembly, the mass of the patch, the arc length through which the patch acceleration assembly travels, and the desired speed of the patch on impact with a surface. The torsion spring constant can be more than about 0.5 Newton*mm/degree, or more than about 2.0 Newton*mm/degree. The torsion spring constant can be less than about 5.0 Newton*mm/degree, or less than about 4.0 Newton*mm/degree. The impactor 62 can be held at various points along the arcuate path either manually or, in some embodiments, with holding means (not shown) that engage and temporarily secure the handle 64 along the slot 48 in the upper cover structure 26 of the housing 22. In some embodiments, demarcations or other indicators (e.g., a force readout display) can be provided for indicating the levels of force associated with particular degrees of displacement of the impactor 62 along the arcuate path.

The range of angular travel of the patch acceleration assembly will often be less than about 170 degrees and sometimes less than about 110 degrees. The range of angular travel of the patch acceleration assembly will often be more than about 10 degrees and sometimes more than about 60 degrees. The mass of the patch acceleration assembly will often be more than about 1 gram and sometimes more than about 5 grams. The mass of the patch acceleration assembly will often be less than about 100 grams and sometimes less than about 30 grams.

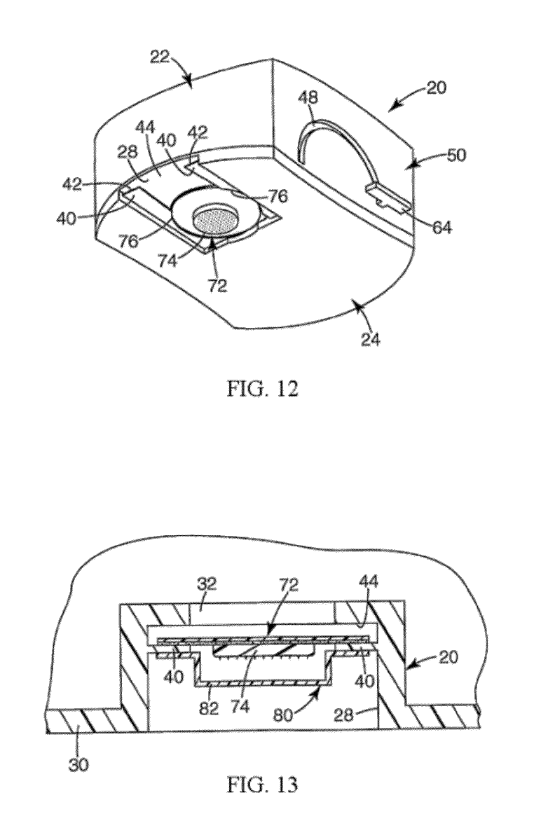

FIG. 12 is a perspective view of a patch 72 (e.g., a patch 72 carrying a microneedle array 74) mounted on the applicator device 20, in accordance with an embodiment of the invention. In an embodiment, the applicator device 20, including the patch 72, is configured to deliver a serotonin receptor antagonist formulation to a subject. In an embodiment, applicator device 20 is configured to deliver an ondansetron-containing formulation or a palonosetron-containing formulation to a subject. The patch 72 is disposed between the retainer members 40 and the bottom portion 44 of the recess 28 in the base 24 of the housing 22. The microneedle array 74 faces away from the opening 32 in the base 24 of the housing 22. The patch 72, which can have adhesive surrounding the microneedle array 74 on the surface facing away from the patch application device 20, contacts the upper surfaces 42 of the retainer members 40, but is generally not adhered firmly to the retainer members 40 due to the release character of the upper surfaces 42. In a fully mounted position, as shown in FIG. 12, microneedle array carried on the patch 72 is generally aligned relative to the opening 32 in the base 24 of the housing 22 (the opening 32 is not visible in FIG. 12).

With continued reference to FIG. 12, the retainer members 40 have cutaway portions 76 that provide an enlarged, partially circular open region that is generally aligned with the opening 32 on the bottom portion 44 of the recess 28 of the base 24 of the housing 22. The wider, open region defined by the cutaway portions 76 facilitates patch application by reducing the amount of deflection of the patch 72 required during deployment to move the patch 72 from a mounted position on the applicator device 20 to a target location. Such cutaway portions 76 can be omitted if, for example, the patch has a generally rectangular shape.

FIG. 13 is a partial cross-sectional view of a microneedle array cartridge 80, having a patch 72 and a cover 82, mounted on an applicator device 20. In an embodiment, the applicator device 20 is similar to the applicator device of FIG. 12. The microneedle array cartridge 80 includes a microneedle array 74. In an embodiment, the microneedle array 74 is configured to deliver serotonin receptor antagonist formulations to a subject. Mounting the patch 72 on the applicator device 20 includes the following steps. The cartridge 80 is partially slid onto the retainer members 40. Then the cartridge 80 is slid further along the retainer members 40, simultaneously separating the cover 82 from the patch 72, until the patch 72 is fully mounted on the applicator device 20 (e.g., such that the microneedle array 74 is aligned with the opening 32 defined in the bottom portion 44 of the recess 28). The cover 82 is removed from (i.e., separated from) the patch 72 to uncover and expose the microneedle array 74 prior to microneedle deployment.

Microinjection methods, devices and systems of embodiments of the invention can be combined or modified with other injection or microinjection methods, devices and systems, including methods, devices and systems for manufacturing microinjection devices and components (such as, e.g., microneedles). For example, the microneedle devices provided herein can be combined or modified with devices, apparatuses, systems and methods (including methods of manufacturing) described in U.S. Patent Publication Nos. 2003/0045837 to Delmore et al., 2003/0135161 to Fleming et al., 2005/0143713 to Delmore et al., 2005/0187521 to Fleming et al., 2005/0261631 to Clarke et al., 2006/0195067 to Wolter et al., 2007/0083151 to Carter, 2007/0191761 to Boone et al., 2008/0009811 to Cantor, 2008/0009825 to Ringsred et al., 2008/0039805 to Frederickson et al., 2008/0051699 to Choi et al., 2008/0088066 to Ferguson et al., 2008/0102192 to Johnson et al., 2008/0108958 to Carter et al., 2008/0114298 to Cantor et al., 2008/0195035 to Frederickson et al., 2008/0208146 to Brandwein et al., 2008/0262416 to Duan et al., 2008/0275400 to Ferguson, 2008/0287858 to Duan, 2008/0294116 to Wolter et al., 2008/0319404 to Pekurovsky et al., 2009/0099537 to DeVoe et al., 2009/0171314 to Ferguson, 2009/0198189 to Simons et al., 2009/0277794 to Trice et al., 2010/0159197 to Ferguson et al., 20100193997 to Frederickson et al. and 2010/0222743 to Frederickson et al., which are entirely incorporated herein by reference, and U.S. Pat. Nos. 6,881,203 to Delmore et al. and 6,908,453 to Fleming et al., which are entirely incorporated herein by reference. As another example, the microneedle (or microinjection) devices provided herein can be combined or modified with devices, apparatuses, systems and methods (such as methods of manufacturing) described in U.S. Patent Publication No. 2004/0249339, U.S. Patent Publication No. 2005/0154350, U.S. Patent Publication No. 2005/0137536, U.S. Patent Publication No. 2003/0135201, U.S. Patent Publication No. 2009/0043250, U.S. Patent Publication No. 2003/0135158, U.S. Patent Publication No. 2003/0135166, U.S. Patent Publication No. 2003/0135167, U.S. Patent Publication No. 2009/0062752, U.S. Patent Publication No. 2005/0119618, U.S. Patent Publication No. 2006/0030838, U.S. Patent Publication No. 2004/0106904, U.S. Patent Publication No. 2009/0118672, U.S. Patent Publication No. 2009/0240232, U.S. Patent Publication No. 2003/0149397, U.S. Patent Publication No. 2002/0156418, U.S. Patent Publication No. 2002/0151842, U.S. Patent Publication No. 2002/0161329, U.S. Patent Publication No. 2009/0157005, U.S. Patent Publication No. 2009/0198185, U.S. Patent Publication No. 2010/0217191, U.S. Patent Publication No. 2002/0169416, U.S. Patent Publication No. 2006/0189939, U.S. Patent Publication No. 2006/0189939, U.S. Patent Publication No. 2009/0093763, U.S. Pat. No. 6,939,324, U.S. Pat. No. 7,150,409, U.S. Pat. No. 7,481,792, U.S. Pat. No. 7,530,968, U.S. Pat. No. 7,187,969, U.S. Pat. No. 6,616,627, U.S. Pat. No. 6,406,455, U.S. Pat. No. 6,314,317, U.S. Pat. No. 6,960,184, U.S. Pat. No. 6,490,483, U.S. Pat. No. 6,939,324, U.S. Pat. No. 7,027,478, U.S. Pat. No. 6,230,051, WO/2000/035520, WO/2001/051109, WO/2002/051470, WO/2002/050584, WO/2003/024507, WO/2003/026732, WO/2004/033021, and WO/2007/115039, which are entirely incorporated herein by reference.

### Methods and Systems for Treating Subjects

In yet another aspect of the invention, microinjection devices are used to deliver serotonin receptor antagonist formulations to subjects. Microinjection devices for delivering serotonin receptor antagonist formulations can be selected from any microinjection or microneedle devices provided herein. In an embodiment, a microinjection device having one or more microneedles is used to deliver a serotonin receptor antagonist formulation to a subject. In another embodiment, a microinjection device having a plurality of microneedles can be used to deliver an ondansetron-containing formulation or palonosetron-containing formulation to a subject. In an embodiment, the ondansetron-containing formulation or palonosetron-containing formulation is delivered to a subject subcutaneously. In another embodiment, the ondansetron-containing formulation or palonosetron-containing formulation is delivered to a subject in a transdermal fashion. In another embodiment, the ondansetron-containing formulation or palonosetron-containing formulation is delivered to a subject in an intradermal fashion.

In an embodiment, a microinjection device having a serotonin receptor antagonist formulation can be used to treat nausea, vomiting or both nausea and vomiting, such as nausea and/or vomiting induced by chemotherapy. In another embodiment, a microinjection device having a serotonin receptor antagonist formulation can be used to treat schizophrenia, sleep apnea, Parkinson's disease, obsessive compulsive disorder, alcoholism, opioid addiction, irritable bowel syndrome and postanesthetic shivering.

In an embodiment, a microinjection device having an ondansetron formulation or palonosetron formulation can be used to treat nausea, vomiting or both nausea and vomiting, such as nausea and/or vomiting induced by chemotherapy. In another embodiment, a microinjection device having an ondansetron formulation or palonosetron formulation can be used to treat schizophrenia, sleep apnea, Parkinson's disease, obsessive compulsive disorder, alcoholism, opioid addiction, irritable bowel syndrome and postanesthetic shivering.

In an embodiment, a serotonin receptor antagonist is administered to a subject by subcutaneous, transdermal or intradermal administration. In another embodiment, subcutaneous, transdermal or intradermal administration is by drug vehicle interaction. In yet another embodiment, subcutaneous, transdermal or intradermal administration is by the use of ion pairs or coacervates. In still another embodiment, subcutaneous, transdermal or intradermal administration is by vesicles and particles. In still another embodiment, subcutaneous, transdermal or intradermal administration is by liposomes and analogues. In still another embodiment, subcutaneous, transdermal or intradermal administration is with the use of high velocity particles. In still another embodiment, subcutaneous, transdermal or intradermal administration is by removing, bypassing or modifying the stratum corneum. In still another embodiment, subcutaneous, transdermal or intradermal administration is by hydration. In still another embodiment, subcutaneous, transdermal or intradermal administration is with the use of chemical enhances. In still another embodiment, subcutaneous, transdermal or intradermal administration is by microneedle injection. In still another embodiment, subcutaneous, transdermal or intradermal administration is by ablation. In still another embodiment, subcutaneous, transdermal or intradermal administration is by follicular delivery. In still another embodiment, subcutaneous, transdermal or intradermal administration is by electrically assisted methods. In still another embodiment, subcutaneous, transdermal or intradermal administration is by ultrasound. In still another embodiment, subcutaneous, transdermal or intradermal administration is by iontophoresis. In still another embodiment, subcutaneous, transdermal or intradermal administration is by electroporation.

In an embodiment, a serotonin receptor antagonist is administered subcutaneously, transdermally or intradermally with the aid of iontophoresis, which can involve non-invasively propelling high concentrations of a charged substance, such as a serotonin receptor antagonist formulation, subcutaneously, transdermally or intradermally by a repulsive electromotive force using a small electrical charge applied to an iontophoretic chamber containing a similarly charged active agent, such as a serotonin receptor antagonist, and its vehicle. In another embodiment, ondansetron or palonosetron is administered subcutaneously, transdermally or intradermally with the aid of iontophoresis. In yet another embodiment, a device having one or more chambers filled with a solution containing a serotonin receptor antagonist is provided. The serotonin receptor antagonist can be provided in the one or more chambers with a solvent to aid in (or facilitate) delivery. The device can include one or both of a positively charged chamber for repelling a positively charged chemical and a negatively charged chamber for repelling a negatively charged chemical into the skin of a subject.

In an embodiment, a serotonin receptor antagonist is administered subcutaneously, transdermally or intradermally with the aid of ultrasound or ultrasonic energy (also “ultrasound” herein). In another embodiment, ondansetron or palonosetron is administered subcutaneously, transdermally or intradermally with the aid of ultrasound. The application of ultrasound to the skin can increase the permeability of skin to a serotonin receptor antagonist, which can enable the delivery of a serotonin receptor antagonist, such as ondansetron or palonosetron, through the skin.

In an embodiment, a serotonin receptor antagonist is administered subcutaneously, transdermally or intradermally with the aid of electroporation. In another embodiment, ondansetron or palonosetron is administered subcutaneously, transdermally or intradermally with the aid of electroporation. In another embodiment, a device is provided for applying an electric field to an area of a subject's body in which transdermal administration of a serotonin receptor antagonist is desired, such as, for example, a portion of a subject's arm. The application of the electric field can facilitate the transdermal delivery of the serotonin receptor antagonist, such as ondansetron or palonosetron, to the subject.

In an embodiment, a serotonin receptor antagonist is administered subcutaneously, transdermally or intradermally by microneedle injection. In another embodiment, ondansetron or palonosetron is administered subcutaneously, transdermally or intradermally by microneedle injection. Microneedle injection can include use of a microneedle device, such as a microneedle device of various embodiments of the invention.

In an embodiment, a first user employs a microinjection device having a serotonin receptor antagonist formulation to deliver the serotonin receptor antagonist formulation to a subject. In an embodiment, the first user is a doctor or healthcare professional and the subject is a patient. In another embodiment, the first user is a caregiver and the second user is a subject under the caregiver's care. In another embodiment, the first user is a friend or relative of the subject.

In another embodiment, a subject employs a microinjection device having a serotonin receptor antagonist formulation to self-administer the serotonin receptor antagonist formulation. In another embodiment, a subject employs a microinjection device having an ondansetron formulation or palonosetron formulation to self-administer the ondansetron formulation or palonosetron formulation, respectively.

It will be appreciated that the term “user”, as used herein, can refer to an individual using a microinjection device to administer a serotonin receptor antagonist formulation to another individual, such as a subject, or to an individual using the microinjection device to administer the serotonin receptor antagonist formulation to her or himself. The term “subject”, as used herein, can refer to an individual under treatment by another individual, such as a healthcare provider (e.g., physician, physician's assistant, nurse) or a care provider, or to an individual administering the serotonin receptor antagonist formulation to himself or herself (i.e., self administration). A “subject” includes asymptomatic individuals and symptomatic individuals, such as a patient.

In an embodiment, a serotonin receptor antagonist formulation (or serotonin receptor antagonist-containing formulation) can have a serotonin receptor antagonist concentration (mg serotonin receptor antagonist/mL formulation) of about 0.001 mg/l mL, or 0.002 mg/l mL, or 0.003 mg/l mL, or 0.004 mg/l mL, or 0.005 mg/l mL, or 0.006 mg/l mL, or 0.007 mg/l mL, or 0.008 mg/l mL, or 0.009 mg/l mL, or 0.01 mg/l mL, or 0.02 mg/l mL, or 0.03 mg/l mL, or 0.04 mg/l mL, or 0.05 mg/l mL, or 0.06 mg/l mL, or 0.07 mg/l mL, or 0.08 mg/l mL, or 0.09 mg/l mL, or 0.1 mg/l mL, or 0.2 mg/l mL, or 0.3 mg/l mL, or 0.4 mg/l mL, or 0.5 mg/l mL, or 0.6 mg/l mL, or 0.7 mg/l mL, or 0.8 mg/l mL, or 0.9 mg/l mL, or 1 mg/l mL, or 2 mg/l mL, or 3 mg/l mL, or 4 mg/l mL, or 5 mg/l mL, or 6 mg/l mL, or 7 mg/l mL, or 8 mg/l mL, or 9 mg/l mL, or 10 mg/l mL, or 11 mg/l mL, or 12 mg/l mL, or 13 mg/l mL, or 14 mg/l mL, or 15 mg/l mL, or 16 mg/l mL, or 17 mg/l mL, or 18 mg/l mL, or 19 mg/l mL, or 20 mg/l mL, or 21 mg/l mL, or 22 mg/l mL, or 23 mg/l mL, or 24 mg/l mL, or 25 mg/l mL, or 26 mg/l mL, or 27 mg/l mL, or 28 mg/l mL, or 29 mg/l mL, or 30 mg/l mL or 31 mg/l mL, or 32 mg/l mL, or 33 mg/l mL, or 34 mg/l mL, or 35 mg/l mL, or 36 mg/l mL, or 37 mg/l mL, or 38 mg/l mL, or 39 mg/l mL, or 40 mg/l mL. In another embodiment, a serotonin receptor antagonist formulation can have a serotonin receptor antagonist concentration between about 0.001 mg/l mL and 40 mg/l mL, or between about 0.01 mg/l mL and 30 mg/l mL, or between about 0.02 mg/l mL and 25 mg/l mL.

In embodiments, the dosing and concentration profiles of serotonin receptor antagonists can vary with the particular type of serotonin receptor antagonist desired for administration to a subject with the aid of a microinjection device. For instance, ondansetron can be administered to a subject at a concentration of about 4 mg/0.5 mL (or 8 mg/l mL), whereas palonosetron can be administered to a subject at a concentration of about 0.075 mg/1.5 mL (or 0.05 mg/l mL).

In an embodiment, an ondansetron formulation (or ondansetron-containing formulation) can have an ondansetron concentration (mg ondansetron/mL formulation) of about 0.01 mg/l mL, or 0.02 mg/l mL, or 0.03 mg/l mL, or 0.04 mg/l mL, or 0.05 mg/l mL, or 0.06 mg/l mL, or 0.07 mg/l mL, or 0.08 mg/l mL, or 0.09 mg/l mL, or 0.1 mg/l mL, or 0.2 mg/l mL, or 0.3 mg/l mL, or 0.4 mg/l mL, or 0.5 mg/l mL, or 0.6 mg/l mL, or 0.7 mg/l mL, or 0.8 mg/l mL, or 0.9 mg/l mL, or 1 mg/l mL, or 2 mg/l mL, or 3 mg/l mL, or 4 mg/l mL, or 5 mg/l mL, or 6 mg/l mL, or 7 mg/l mL, or 8 mg/l mL, or 9 mg/l mL, or 10 mg/l mL, or 11 mg/l mL, or 12 mg/l mL, or 13 mg/l mL, or 14 mg/l mL, or 15 mg/l mL, or 16 mg/l mL, or 17 mg/l mL, or 18 mg/l mL, or 19 mg/l mL, or mg/l mL, or 21 mg/l mL, or 22 mg/l mL, or 23 mg/l mL, or 24 mg/l mL, or 25 mg/l mL, or 26 mg/l mL, or 27 mg/l mL, or 28 mg/l mL, or 29 mg/l mL, or 30 mg/l mL or 31 mg/l mL, or 32 mg/l mL, or 33 mg/l mL, or 34 mg/l mL, or 35 mg/l mL, or 36 mg/l mL, or 37 mg/l mL, or 38 mg/l mL, or 39 mg/l mL, or 40 mg/l mL. In another embodiment, an ondansetron formulation can have an ondansetron concentration between about 0.01 mg/l mL and 40 mg/l mL, or between about 1 mg/l mL and 30 mg/l mL, or between about 5 mg/l mL and 25 mg/l mL.

In an embodiment, a palonosetron formulation (or palonosetron-containing formulation) can have a palonosetron concentration (mg palonosetron/mL formulation) of about 0.001 mg/l mL, or 0.002 mg/l mL, or 0.003 mg/l mL, or 0.004 mg/l mL, or 0.005 mg/l mL, or 0.006 mg/l mL, or 0.007 mg/l mL, or 0.008 mg/l mL, or 0.009 mg/l mL, or 0.01 mg/l mL, or 0.015 mg/l mL, or 0.02 mg/l mL, or 0.025 mg/l mL, or 0.03 mg/l mL, or 0.035 mg/l mL, or 0.04 mg/l mL, or 0.045 mg/l mL, or 0.05 mg/l mL, or 0.055 mg/l mL, or 0.06 mg/l mL, or 0.065 mg/l mL, or 0.07 mg/l mL, or 0.075 mg/l mL, or 0.08 mg/l mL, or 0.085 mg/l mL, or 0.09 mg/l mL, or 0.095 mg/l mL, or 0.1 mg/l mL, or 0.2 mg/l mL, or 0.3 mg/l mL, or 0.4 mg/l mL, or 0.5 mg/l mL, or 0.6 mg/l mL, or 0.7 mg/l mL, or 0.8 mg/l mL, or 0.9 mg/l mL, or 1 mg/l mL. In another embodiment, a palonosetron formulation can have a palonosetron concentration between about 0.001 mg/l mL and 1 mg/l mL, or between about 0.01 mg/l mL and 0.1 mg/l mL, or between about 0.02 mg/l mL and 0.07 mg/l mL. In another embodiment, a palonosetron formulation can have a palonosetron concentration between about 0.0015 mg/1.5 mL and 1.5 mg/1.5 mL, or between about 0.015 mg/1.5 mL and 0.15 mg/1.5 mL, or between about 0.03 mg/1.5 mL and 0.105 mg/1.5 mL.

In an embodiment, a microinjection device, such as any device provided herein, is used to deliver ondansetron or palonosetron to a subject from once a day to once a month or more. In another embodiment, a microinjection device, such as any device provided herein, is used to deliver ondansetron or palonosetron to a subject from once a day to once a week. In another embodiment, a microinjection device, such as any device provided herein, is used to deliver ondansetron, palonosetron or other serotonin receptor antagonist to a subject at least once a day, once every two days, once every three days, once every four days, once every five days, once every six days, once a week, once every two weeks, once every three weeks, once a month, once every two months, once every three months, once every four months, once every five months, once every six months, once a year, or more. In another embodiment, a microinjection device, such as any device provided herein, is used to deliver ondansetron, palonosetron or other serotonin receptor antagonist to a subject at least once a day, or twice a day, or three times per day, or four times per day, or five times per day, or six times per day, or seven times per day, or eight times per day, or nine times per day, or ten times per day, or eleven times per day, or twelve times per day, or thirteen times per day, or fourteen times per day, or fifteen times per day, or sixteen times per day, or seventeen times per day, or eighteen times per day, or nineteen times per day, or twenty times per day, or twenty one times per day, or twenty two times per day, or twenty three times per day, or twenty four times per day.

In an embodiment, a microinjection device is used to deliver a serotonin receptor antagonist or a serotonin receptor antagonist formulation to a subject at a dose of at least about 0.01 mg, or 0.02 mg, or 0.03 mg, or 0.04 mg, or 0.05 mg, or 0.06 mg, or 0.07 mg, or 0.08 mg, or 0.09 mg, or 0.1 mg, or 0.2 mg, or 0.3 mg, or 0.4 mg, or 0.5 mg, or 0.6 mg, or 0.7 mg, or 0.8 mg, or 0.9 mg, or 1 mg, or 2 mg, or 3 mg, or 4 mg, or 5 mg, or 6 mg, or 7 mg, or 8 mg, or 9 mg, or 10 mg, or 11 mg, or 12 mg, or 13 mg, or 14 mg, or 15 mg, or 16 mg, or 17 mg, or 18 mg, or 19 mg, or 20 mg, or 30 mg, or 40 mg, or 50 mg. In another embodiment, a microinjection device is used to deliver ondansetron or an ondansetron formulation to a subject at a dose of at least about 0.01 mg, or 0.02 mg, or 0.03 mg, or 0.04 mg, or 0.05 mg, or 0.06 mg, or 0.07 mg, or 0.08 mg, or 0.09 mg, or 0.1 mg, or 0.2 mg, or 0.3 mg, or 0.4 mg, or 0.5 mg, or 0.6 mg, or 0.7 mg, or 0.8 mg, or 0.9 mg, or 1 mg, or 2 mg, or 3 mg, or 4 mg, or 5 mg, or 6 mg, or 7 mg, or 8 mg, or 9 mg, or 10 mg, or 11 mg, or 12 mg, or 13 mg, or 14 mg, or 15 mg, or 16 mg, or 17 mg, or 18 mg, or 19 mg, or 20 mg, or 30 mg, or 40 mg, or 50 mg. In another embodiment, a microinjection device is used to deliver palonosetron or a palonosetron formulation to a subject at a dose of at least about 0.001 mg, or 0.002 mg, or 0.003 mg, or 0.004 mg, or 0.005 mg, or 0.006 mg, or 0.007 mg, or 0.008 mg, or 0.009 mg, or 0.01 mg, or 0.015 mg, or 0.02 mg, or 0.025 mg, or 0.03 mg, or 0.035 mg, or 0.04 mg, or 0.045 mg, or 0.05 mg, or 0.055 mg, or 0.06 mg, or 0.065 mg, or 0.07 mg, or 0.075 mg, or 0.08 mg, or 0.085 mg, or 0.09 mg, or 0.095 mg, or 0.1 mg, or 0.15 mg, 0.2 mg, or 0.25 mg, or 0.3 mg, or 0.35 mg, or 0.4 mg, or 0.45 mg, or 0.5 mg, or 0.55 mg, or 0.6 mg, or 0.65 mg, or 0.7 mg, or 0.75 mg, or 0.8 mg, or 0.85 mg, or 0.9 mg, or 0.95 mg, or 1 mg, or 2 mg, or 3 mg, or 4 mg, or 5 mg.

In an embodiment, a microinjection device is used to deliver a serotonin receptor antagonist or a serotonin receptor antagonist formulation to a subject at a regimen (volume) of at least about 0.1 mL, or 0.2 mL, or 0.3 mL, or 0.4 mL, or 0.5 mL, or 0.6 mL, or 0.7 mL, or 0.8 mL, or 0.9 mL, or 1.0 mL, or 1.1 mL, or 1.2 mL, or 1.3 mL, or 1.4 mL, or 1.5 mL, or 1.6 mL, or 1.7 mL, or 1.8 mL, or 1.9 mL, or 2.0 mL, or 3.0 mL, or 4.0 mL, or 5.0 mL, or more. In another embodiment, a microinjection device is used to deliver ondansetron or an ondansetron formulation to a subject at a regimen (volume) of at least about 0.1 mL, or 0.2 mL, or 0.3 mL, or 0.4 mL, or 0.5 mL, or 0.6 mL, or 0.7 mL, or 0.8 mL, or 0.9 mL, or 1.0 mL, or 1.1 mL, or 1.2 mL, or 1.3 mL, or 1.4 mL, or 1.5 mL, or 1.6 mL, or 1.7 mL, or 1.8 mL, or 1.9 mL, or 2.0 mL, or 3.0 mL, or 4.0 mL, or 5.0 mL, or more. In another embodiment, a microinjection device is used to deliver palonosetron or a palonosetron formulation to a subject at a regimen (volume) of at least about 0.1 mL, or 0.2 mL, or 0.3 mL, or 0.4 mL, or 0.5 mL, or 0.6 mL, or 0.7 mL, or 0.8 mL, or 0.9 mL, or 1.0 mL, or 1.1 mL, or 1.2 mL, or 1.3 mL, or 1.4 mL, or 1.5 mL, or 1.6 mL, or 1.7 mL, or 1.8 mL, or 1.9 mL, or 2.0 mL, or 3.0 mL, or 4.0 mL, or 5.0 mL, or more.

In an embodiment, a microinjection device is used to deliver a serotonin receptor antagonist to a subject once a day at a dosage (or dose) of about 0.1 milligram (“mg”) or less, or 0.2 mg or less, or 0.3 mg or less, or 0.4 mg or less, or 0.5 mg or less, or 1 mg or less, or 2 mg or less, or 3 mg or less, or 4 mg or less, or 5 mg or less, or 6 mg or less, or 7 mg or less, or 8 mg or less, or 9 mg or less, or 10 mg or less, or 11 mg or less, or 12 mg or less, or 13 mg or less, or 14 mg or less, or mg or less, or 16 mg or less, or 17 mg or less, or 18 mg or less, or 19 mg or less, or 20 mg or less, or 21 mg or less, or 22 mg or less, or 23 mg or less, or 24 mg or less, or 25 mg or less, or 30 mg or less, or 40 mg or less, or 50 mg or less. In another embodiment, a microinjection device is used to deliver a serotonin receptor antagonist to a subject once a day at a dosage of about 20 mg in a 1 mL formulation having the serotonin receptor antagonist.

Unless the context indicates otherwise, formulation volumes, when used in association with doses (mg), are used to illustrate concentrations and may not necessarily be the volumes of formulations delivered to subjects. In an example, a microinjection device is loaded with about 0.5 mL of a serotonin receptor antagonist formulation having a serotonin receptor antagonist concentration of about 21 mg in 1 mL.

In an embodiment, a microinjection device is used to deliver a serotonin receptor antagonist to a subject at a dose up to an including about 0.1 mg/day, or 0.15 mg/day, or 0.2 mg/day, or 0.25 mg/day, or 0.3 mg/day, or 0.35 mg/day, or 0.4 mg/day, or 0.45 mg/day, or 0.5 mg/day, or 0.55 mg/day, or 0.6 mg/day, or 0.65 mg/day, or 0.7 mg/day, or 0.75 mg/day, or 0.8 mg/day, or 0.85 mg/day, or 0.9 mg/day, or 0.95 mg/day, or 1 mg/day, or 2 mg/day, or 3 mg/day, or 4 mg/day, or 5 mg/day, or 6 mg/day, or 7 mg/day, or 8 mg/day, or 9 mg/day, or 10 mg/day, or 11 mg/day, or 12 mg/day, or 13 mg/day, or 14 mg/day, or 15 mg/day, or 16 mg/day, or 17 mg/day, or 18 mg/day, or 19 mg/day, or 20 mg/day, or 21 mg/day, or 22 mg/day, or 23 mg/day, or 24 mg/day, or 25 mg/day, or 26 mg/day, or 27 mg/day, or 28 mg/day, or 29 mg/day, or 30 mg/day, or 31 mg/day, or 32 mg/day, or 33 mg/day, or 34 mg/day, or 35 mg/day, or 36 mg/day, or 37 mg/day, or 38 mg/day, or 39 mg/day, or 40 mg/day.

In an embodiment, a microinjection device is used to deliver ondansetron to a subject once a day at a dosage of about 1 milligram (“mg”) or less, or 2 mg or less, or 3 mg or less, or 4 mg or less, or 5 mg or less, or 6 mg or less, or 7 mg or less, or 8 mg or less, or 9 mg or less, or 10 mg or less, or 11 mg or less, or 12 mg or less, or 13 mg or less, or 14 mg or less, or 15 mg or less, or 16 mg or less, or 17 mg or less, or 18 mg or less, or 19 mg or less, or 20 mg or less, or 21 mg or less, or 22 mg or less, or 23 mg or less, or 24 mg or less, or 25 mg or less, or 30 mg or less, or 40 mg or less, or 50 mg or less. In another embodiment, a microinjection device is used to deliver ondansetron to a subject once a day at a dosage of about 8 mg in a 1 mL formulation having ondansetron, or 4 mg in a 0.5 mL formulation having ondansetron.

In an embodiment, a microinjection device is used to deliver ondansetron to a subject at a dose up to an including about 1 mg/day, or 2 mg/day, or 3 mg/day, or 4 mg/day, or 5 mg/day, or 6 mg/day, or 7 mg/day, or 8 mg/day, or 9 mg/day, or 10 mg/day, or 11 mg/day, or 12 mg/day, or 13 mg/day, or 14 mg/day, or 15 mg/day, or 16 mg/day, or 17 mg/day, or 18 mg/day, or 19 mg/day, or mg/day, or 21 mg/day, or 22 mg/day, or 23 mg/day, or 24 mg/day, or 25 mg/day, or 26 mg/day, or 27 mg/day, or 28 mg/day, or 29 mg/day, or 30 mg/day, or 31 mg/day, or 32 mg/day, or 33 mg/day, or 34 mg/day, or 35 mg/day, or 36 mg/day, or 37 mg/day, or 38 mg/day, or 39 mg/day, or 40 mg/day.

In an embodiment, a microinjection device is used to deliver palonosetron to a subject once a day at a dosage of about 0.001 milligram (“mg”) or less, or 0.002 mg or less, or 0.003 mg or less, or 0.004 mg or less, or 0.005 mg or less, or 0.006 mg or less, or 0.007 mg or less, or 0.008 mg or less, or 0.009 mg or less, or 0.01 mg or less, or 0.02 mg or less, or 0.03 mg or less, or 0.04 mg or less, or 0.05 mg or less, or 0.06 mg or less, or 0.07 mg or less, or 0.08 mg or less, or 0.09 mg or less, or 0.1 mg or less, or 0.15 mg or less, or 0.2 mg or less, or 0.25 mg or less, or 0.3 mg or less, or 0.35 mg or less, or 0.4 mg or less, or 0.45 mg or less, or 0.5 mg or less, or 0.55 mg or less, or 0.6 mg or less, or 0.65 mg or less, or 0.7 mg or less, or 0.75 mg or less, or 0.8 mg or less, or 0.85 mg or less, or 0.9 mg or less, or 0.95 mg or less, or 1 mg or less. In another embodiment, a microinjection device is used to deliver palonosetron to a subject once a day at a dosage of about 0.05 mg in a 1 mL formulation having palonosetron, or 0.075 mg in a 1.5 mL formulation having palonosetron.

In an embodiment, a microinjection device is used to deliver palonosetron to a subject at a dose up to an including about 0.001 mg/day, or 0.002 mg/day, or 0.003 mg/day, or 0.004 mg/day, or 0.005 mg/day, or 0.006 mg/day, or 0.007 mg/day, or 0.008 mg/day, or 0.009 mg/day, or 0.01 mg/day, or 0.02 mg/day, or 0.03 mg/day, or 0.04 mg/day, or 0.05 mg/day, or 0.06 mg/day, or 0.07 mg/day, or 0.08 mg/day, or 0.09 mg/day, or 0.1 mg/day, or 0.15 mg/day, or 0.2 mg/day, or 0.25 mg/day, or 0.3 mg/day, or 0.35 mg/day, or 0.4 mg/day, or 0.45 mg/day, or 0.5 mg/day, or 0.55 mg/day, or 0.6 mg/day, or 0.65 mg/day, or 0.7 mg/day, or 0.75 mg/day, or 0.8 mg/day, or 0.85 mg/day, or 0.9 mg/day, or 0.95 mg/day, or 1 mg/day.

In an embodiment, the length of time in which a given dosage of a serotonin receptor antagonist is delivered to a subject using a microinjection device is dependent on various fluid and delivery properties, such as the volume of a serotonin receptor antagonist formulation, the viscosity of the formulation, the flow rate of the formulation from the microinjection device, the diameter of any fluid channels in any microneedles included in the microinjection device, and the pressure drop across fluid channels in any hollow microneedles included in the microinjection device. In an embodiment, a microinjection device can be used to deliver a serotonin receptor antagonist formulation to a subject in a time period between about 0.1 seconds and 60 minutes, or between about 30 seconds and 30 minutes, or between about 1 minute and 7 minutes, or between about 2 minutes and 6 minutes, or between about 3 minutes and 5 minutes. In another embodiment, a microinjection device can be used to deliver a serotonin receptor antagonist formulation to a subject in a time period up to an including 1 minute, or 2 minutes, or 3 minutes, or 4 minutes, or 5 minutes, or 6 minutes, or 7 minutes, or 8 minutes, or 9 minutes, or 10 minutes, or 30 minutes, or 1 hour, or 2 hours, or 3 hours, or 4 hours, or 5 hours, or 6 hours, or 7 hours, or 8 hours, or 9 hours, or 10 hours, or hours, or 20 hours, or 24 hours.

In an embodiment, the length of time in which a given dosage of ondansetron or palonosetron (or other serotonin receptor antagonist) is delivered to a subject using a microinjection device is dependent on various fluid and delivery properties, such as the volume of an ondansetron-containing formulation or palonosetron-containing formulation, the viscosity of the formulation, the flow rate of the formulation from the microinjection device, the diameter of any fluid channels in any microneedles included in the microinjection device, and the pressure drop across fluid channels in any microneedles included in the microinjection device. In an embodiment, a microinjection device can be used to deliver an ondansetron formulation or a or palonosetron formulation to a subject in a time period between about 0.1 seconds and 60 minutes, or between about 30 seconds and 30 minutes, or between about 1 minute and 7 minutes, or between about 2 minutes and 6 minutes, or between about 3 minutes and 5 minutes. In another embodiment, a microinjection device can be used to deliver an ondansetron formulation or a palonosetron formulation to a subject in a time period up to an including 1 minute, or 2 minutes, or 3 minutes, or 4 minutes, or 5 minutes, or 6 minutes, or 7 minutes, or 8 minutes, or 9 minutes, or 10 minutes, or 30 minutes, or 1 hour, or 2 hours, or 3 hours, or 4 hours, or 5 hours, or 6 hours, or 7 hours, or 8 hours, or 9 hours, or 10 hours, or hours, or 20 hours, or 24 hours.

In an embodiment, a microinjection device, such as any device provided herein, can be used to deliver a serotonin receptor antagonist to a subject at a dosage, in a 1 mL formulation of the serotonin receptor antagonist, of at least about 1 mg, or 2 mg, or 3 mg, or 4 mg, or 5 mg, or 6 mg, or 7 mg, or 8 mg, or 9 mg, or 10 mg, or 11 mg, or 12 mg, or 13 mg, or 14 mg, or 15 mg, or 16 mg, or 17 mg, or 18 mg, or 19 mg, or 20 mg, or 21 mg, or 22 mg, or 23 mg, or 24 mg, or 25 mg, or 26 mg, or 27 mg, or 28 mg, or 29 mg, or 30 mg, or 31 mg, or 32 mg, or 33 mg, or 34 mg, or 35 mg, or 36 mg, or 37 mg, or 38 mg, or 39 mg, or 40 mg, or 50 mg. In another embodiment, a microinjection device, such as any device provided herein, can be used to deliver a serotonin receptor antagonist to a subject every 0.1 hours, or 0.2 hours, or 0.3 hours, or 0.4 hours, or 0.5 hours, or 1 hour, or 2 hours, or 3 hours, or 4 hours, or 5 hours, or 6 hours, or 7 hours, or 8 hours, or 9 hours, or 10 hours, or 11 hours, or 12 hours, or once a day, or more. In another embodiment, a microinjection device, such as any device provided herein, can be used to deliver a serotonin receptor antagonist to a subject over a time period of about 0.1 seconds to 60 minutes, or about 1 second to 30 minutes, or about 5 seconds to 5 minutes, or about 10 seconds to 1 minute, or about 15 to 45 seconds. In an embodiment, a serotonin receptor antagonist can be delivered to a subject over a period of about 1 minute or less, or 2 minutes or less, or 3 minutes or less, or 4 minutes or less, or 5 minutes or less, or 6 minutes or less, or 7 minutes or less, or 8 minutes or less, or 9 minutes or less, or 10 minutes or less, or 30 minutes or less, or 1 hour or less, or 2 hours or less, or 3 hours or less, or 4 hours or less, or 5 hours or less, or 6 hours or less, or 7 hours or less, or 8 hours or less, or 9 hours or less, or 10 hours or less, or 11 hours or less, or 12 hours or less, or 13 hours or less, or 14 hours or less, or 15 hours or less, or 16 hours or less, or 17 hours or less, or 18 hours or less, or 19 hours or less, or 20 hours or less, or 21 hours or less, or 22 hours or less, or 23 hours or less, or 24 hours or less. In another embodiment, a microinjection device, such as any device provided herein, can be used to deliver a serotonin receptor antagonist to a subject at a dosage of about 20 mg in a 1 mL formulation once a day over a period less than about 1 hour, or less than about 30 minutes, or less than about 10 minutes, or less than about 1 minute. In another embodiment, a microinjection device, such as any device provided herein, can be used to deliver a serotonin receptor antagonist to a subject in a time period of at least about 30 seconds, or 1 minute, or 1.5 minutes, or 2 minutes, or 2.5 minutes, or 3 minutes, or 3.5 minutes, or 4 minutes, or 4.5 minutes, or 5 minutes, or 5.5 minutes, or 6 minutes, or 7.5 minutes, or 8 minutes, or 8.5 minutes, or 9 minutes, or 9.5 minutes, or 10 minutes, or 10.5 minutes, or 11 minutes, or 11.5 minutes, or 12 minutes, or 12.5 minutes, or 13 minutes, or 13.5 minutes, or 14 minutes, or 14.5 minutes, or 15 minutes, or 15.5 minutes, or 16 minutes, or 16.5 minutes, or 17 minutes, or 17.5 minutes, or 18 minutes, or 18.5 minutes, or 19 minutes, or 19.5 minutes, or 20 minutes, or 20.5 minutes, or 21 minutes, or 21.5 minutes, or 22 minutes, or 22.5 minutes, or 23 minutes, or 23.5 minutes, or 24 minutes, or 24.5 minutes, or 25 minutes, or 25.5 minutes, or 26 minutes, or 26.5 minutes, or 27 minutes, or 27.5 minutes, or 28 minutes, or 28.5 minutes, or 29 minutes, or 29.5 minutes, or 30 minutes, or 30.5 minutes, or 31 minutes, or 31.5 minutes, or 32 minutes, or 32.5 minutes, or 33 minutes, or 33.5 minutes, or 34 minutes, or 34.5 minutes, or 35 minutes, or 35.5 minutes, or 36 minutes, or 36.5 minutes, or 37 minutes, or 37.5 minutes, or 38 minutes, or 38.5 minutes, or 39 minutes, or 39.5 minutes, or 40 minutes, or 40.5 minutes, or 41 minutes, or 41.5 minutes, or 42 minutes, or 42.5 minutes, or 43 minutes, or 43.5 minutes, or 44 minutes, or 44.5 minutes, or 45 minutes, or 45.5 minutes, or 46 minutes, or 46.5 minutes, or 47 minutes, or 47.5 minutes, or 48 minutes, or 48.5 minutes, or 49 minutes, or 49.5 minutes, or 50 minutes, or 50.5 minutes, or 51 minutes, or 51.5 minutes, or 52 minutes, or 52.5 minutes, or 53 minutes, or 53.5 minutes, or 54 minutes, or 54.5 minutes, or 55 minutes, or 55.5 minutes, or 56 minutes, or 56.5 minutes, or 57 minutes, or 57.5 minutes, or 58 minutes, or 58.5 minutes, or 59 minutes, or 59.5 minutes, or 60 minutes.

In an embodiment, a microinjection device, such as any device provided herein, can be used to deliver ondansetron to a subject at a dosage, in a 1 mL formulation having the ondansetron, of at least about 1 mg, or 2 mg, or 3 mg, or 4 mg, or 5 mg, or 6 mg, or 7 mg, or 8 mg, or 9 mg, or 10 mg, or 11 mg, or 12 mg, or 13 mg, or 14 mg, or 15 mg, or 16 mg, or 17 mg, or 18 mg, or 19 mg, or mg, or 21 mg, or 22 mg, or 23 mg, or 24 mg, or 25 mg, or 26 mg, or 27 mg, or 28 mg, or 29 mg, or 30 mg, or 31 mg, or 32 mg, or 33 mg, or 34 mg, or 35 mg, or 36 mg, or 37 mg, or 38 mg, or 39 mg, or 40 mg, or 50 mg. In another embodiment, a microinjection device, such as any device provided herein, can be used to deliver ondansetron to a subject every 0.1 hours, or 0.2 hours, or 0.3 hours, or 0.4 hours, or 0.5 hours, or 1 hour, or 2 hours, or 3 hours, or 4 hours, or 5 hours, or 6 hours, or 7 hours, or 8 hours, or 9 hours, or 10 hours, or 11 hours, or 12 hours, or once a day, or more. In another embodiment, a microinjection device, such as any device provided herein, can be used to deliver ondansetron to a subject over a time period of about 0.1 seconds to 60 minutes, or about 1 second to 30 minutes, or about 5 seconds to 5 minutes, or about 10 seconds to 1 minute, or about 15 to 45 seconds. In another embodiment, a microinjection device, such as any device provided herein, can be used to deliver ondansetron to a subject in a time period of about 30 seconds, or 1 minute, or 1.5 minutes, or 2 minutes, or 2.5 minutes, or 3 minutes, or 3.5 minutes, or 4 minutes, or 4.5 minutes, or 5 minutes, or 5.5 minutes, or 6 minutes, or 7.5 minutes, or 8 minutes, or 8.5 minutes, or 9 minutes, or 9.5 minutes, or 10 minutes, or 10.5 minutes, or 11 minutes, or 11.5 minutes, or 12 minutes, or 12.5 minutes, or 13 minutes, or 13.5 minutes, or 14 minutes, or 14.5 minutes, or 15 minutes, or 15.5 minutes, or 16 minutes, or 16.5 minutes, or 17 minutes, or 17.5 minutes, or 18 minutes, or 18.5 minutes, or 19 minutes, or 19.5 minutes, or 20 minutes, or 20.5 minutes, or 21 minutes, or 21.5 minutes, or 22 minutes, or 22.5 minutes, or 23 minutes, or 23.5 minutes, or 24 minutes, or 24.5 minutes, or 25 minutes, or 25.5 minutes, or 26 minutes, or 26.5 minutes, or 27 minutes, or 27.5 minutes, or 28 minutes, or 28.5 minutes, or 29 minutes, or 29.5 minutes, or 30 minutes, or 30.5 minutes, or 31 minutes, or 31.5 minutes, or 32 minutes, or 32.5 minutes, or 33 minutes, or 33.5 minutes, or 34 minutes, or 34.5 minutes, or 35 minutes, or 35.5 minutes, or 36 minutes, or 36.5 minutes, or 37 minutes, or 37.5 minutes, or 38 minutes, or 38.5 minutes, or 39 minutes, or 39.5 minutes, or 40 minutes, or 40.5 minutes, or 41 minutes, or 41.5 minutes, or 42 minutes, or 42.5 minutes, or 43 minutes, or 43.5 minutes, or 44 minutes, or 44.5 minutes, or 45 minutes, or 45.5 minutes, or 46 minutes, or 46.5 minutes, or 47 minutes, or 47.5 minutes, or 48 minutes, or 48.5 minutes, or 49 minutes, or 49.5 minutes, or 50 minutes, or 50.5 minutes, or 51 minutes, or 51.5 minutes, or 52 minutes, or 52.5 minutes, or 53 minutes, or 53.5 minutes, or 54 minutes, or 54.5 minutes, or 55 minutes, or 55.5 minutes, or 56 minutes, or 56.5 minutes, or 57 minutes, or 57.5 minutes, or 58 minutes, or 58.5 minutes, or 59 minutes, or 59.5 minutes, or 60 minutes.

In an embodiment, a microinjection device, such as any device provided herein, can be used to deliver palonosetron to a subject at a dosage, in a 1 mL formulation having the palonosetron, of about 0.001 mg, or 0.002 mg, or 0.003 mg, or 0.004 mg, or 0.005 mg, or 0.006 mg, or 0.007 mg, or 0.008 mg, or 0.009 mg, or 0.01 mg, or 0.02 mg, or 0.03 mg, or 0.04 mg, or 0.05 mg, or 0.06 mg, or 0.07 mg, or 0.08 mg, or 0.09 mg, or 0.1 mg, or 0.15 mg, or 0.2 mg, or 0.25 mg, or 0.3 mg, or 0.35 mg, or 0.4 mg, or 0.45 mg, or 0.5 mg, or 0.55 mg, or 0.6 mg, or 0.65 mg, or 0.7 mg, or 0.75 mg, or 0.8 mg, or 0.85 mg, or 0.9 mg, or 0.95 mg, or 1 mg. In another embodiment, a microinjection device, such as any device provided herein, can be used to deliver palonosetron to a subject every 0.1 hours, or 0.2 hours, or 0.3 hours, or 0.4 hours, or 0.5 hours, or 1 hour, or 2 hours, or 3 hours, or 4 hours, or 5 hours, or 6 hours, or 7 hours, or 8 hours, or 9 hours, or 10 hours, or 11 hours, or 12 hours, or once a day, or more. In another embodiment, a microinjection device, such as any device provided herein, can be used to deliver palonosetron to a subject over a time period of about 0.1 seconds to 60 minutes, or about 1 second to 30 minutes, or about 5 seconds to 5 minutes, or about 10 seconds to 1 minute, or about 15 to 45 seconds. In another embodiment, a microinjection device, such as any device provided herein, can be used to deliver palonosetron to a subject in a time period of about 30 seconds, or 1 minute, or 1.5 minutes, or 2 minutes, or 2.5 minutes, or 3 minutes, or 3.5 minutes, or 4 minutes, or 4.5 minutes, or 5 minutes, or 5.5 minutes, or 6 minutes, or 7.5 minutes, or 8 minutes, or 8.5 minutes, or 9 minutes, or 9.5 minutes, or 10 minutes, or 10.5 minutes, or 11 minutes, or 11.5 minutes, or 12 minutes, or 12.5 minutes, or 13 minutes, or 13.5 minutes, or 14 minutes, or 14.5 minutes, or 15 minutes, or 15.5 minutes, or 16 minutes, or 16.5 minutes, or 17 minutes, or 17.5 minutes, or 18 minutes, or 18.5 minutes, or 19 minutes, or 19.5 minutes, or 20 minutes, or 20.5 minutes, or 21 minutes, or 21.5 minutes, or 22 minutes, or 22.5 minutes, or 23 minutes, or 23.5 minutes, or 24 minutes, or 24.5 minutes, or 25 minutes, or 25.5 minutes, or 26 minutes, or 26.5 minutes, or 27 minutes, or 27.5 minutes, or 28 minutes, or 28.5 minutes, or 29 minutes, or 29.5 minutes, or 30 minutes, or 30.5 minutes, or 31 minutes, or 31.5 minutes, or 32 minutes, or 32.5 minutes, or 33 minutes, or 33.5 minutes, or 34 minutes, or 34.5 minutes, or 35 minutes, or 35.5 minutes, or 36 minutes, or 36.5 minutes, or 37 minutes, or 37.5 minutes, or 38 minutes, or 38.5 minutes, or 39 minutes, or 39.5 minutes, or 40 minutes, or 40.5 minutes, or 41 minutes, or 41.5 minutes, or 42 minutes, or 42.5 minutes, or 43 minutes, or 43.5 minutes, or 44 minutes, or 44.5 minutes, or 45 minutes, or 45.5 minutes, or 46 minutes, or 46.5 minutes, or 47 minutes, or 47.5 minutes, or 48 minutes, or 48.5 minutes, or 49 minutes, or 49.5 minutes, or 50 minutes, or 50.5 minutes, or 51 minutes, or 51.5 minutes, or 52 minutes, or 52.5 minutes, or 53 minutes, or 53.5 minutes, or 54 minutes, or 54.5 minutes, or 55 minutes, or 55.5 minutes, or 56 minutes, or 56.5 minutes, or 57 minutes, or 57.5 minutes, or 58 minutes, or 58.5 minutes, or 59 minutes, or 59.5 minutes, or 60 minutes.

In an embodiment, ondansetron or palonosetron can be delivered to a subject over a period of 1 minute or less, or 2 minutes or less, or 3 minutes or less, or 4 minutes or less, or 5 minutes or less, or 6 minutes or less, or 7 minutes or less, or 8 minutes or less, or 9 minutes or less, or 10 minutes or less, or 30 minutes or less, or 1 hour or less, or 2 hours or less, or 3 hours or less, or 4 hours or less, or 5 hours or less, or 6 hours or less, or 7 hours or less, or 8 hours or less, or 9 hours or less, or 10 hours or less, or 11 hours or less, or 12 hours or less, or 13 hours or less, or 14 hours or less, or 15 hours or less, or 16 hours or less, or 17 hours or less, or 18 hours or less, or 19 hours or less, or 20 hours or less, or 21 hours or less, or 22 hours or less, or 23 hours or less, or 24 hours or less. In another embodiment, a microinjection device, such as any device provided herein, can be used to deliver ondansetron to a subject at a dosage of about 1 mg, or 2 mg, or 3 mg, or 4 mg, or 5 mg, or 6 mg, or 7 mg, or 8 mg, or 9 mg, or 10 mg, or 15 mg, or 20 mg, or 25 mg, or 30 mg, or 35 mg, or 40 mg in a 1 mL formulation once a day over a period less than about 1 hour, or less than about 30 minutes, or less than about 10 minutes, or less than about 1 minute. In another embodiment, a microinjection device, such as any device provided herein, can be used to deliver palonosetron to a subject at a dosage of about 0.01 mg, or 0.02 mg, or 0.03 mg, or 0.04 mg, or 0.05 mg, or 0.06 mg, or 0.07 mg, or 0.08 mg, or 0.09 mg, or 0.1 mg, or 0.15 mg, or 0.2 mg, or 0.25 mg, or 0.3 mg, or 0.35 mg, or 0.4 mg, or 0.45 mg, or 0.5 mg in a 1 mL formulation once a day over a period less than about 1 hour, or less than about 30 minutes, or less than about 10 minutes, or less than about 1 minute.

In an embodiment, the equivalent of 1 mg, or 2 mg, or 3 mg, or 4 mg, or 5 mg, or 6 mg, or 7 mg, or 8 mg, or 9 mg, or 10 mg, or 15 mg, or 20 mg, or 25 mg, or 30 mg, or 35 mg, or 40 mg of ondansetron in a 1 mL solution is delivered to a subject once a day. In another embodiment, 1 mg, or 2 mg, or 3 mg, or 4 mg, or 5 mg, or 6 mg, or 7 mg, or 8 mg, or 9 mg, or 10 mg, or 15 mg, or 20 mg, or 25 mg, or 30 mg, or 35 mg, or 40 mg of ondansetron in a 1 mL solution is delivered to a subject once a day and over a time period up to and including 1 minute, or 2 minutes, or 3 minutes, or 4 minutes, or 5 minutes, or 6 minutes, or 7 minutes, or 8 minutes, or 9 minutes, or 10 minutes, or 20 minutes, or 30 minutes, or 40 minutes, or 50 minutes, or 1 hour, or 2 hours, or 3 hours, or 4 hours, or 5 hours, or 6 hours, or 7 hours, or 8 hours, or 9 hours, or 10 hours, or 11 hours, or 12 hours, or 13 hours, or 14 hours, or 15 hours, or 16 hours, or 17 hours, or 18 hours, or 19 hours, or 20 hours, or 21 hours, or 22 hours, or 23 hours, or 24 hours. In an embodiment, a microinjection device, such as any device provided herein, is loaded with an ondansetron formulation having a concentration of about 1 mg/l mL, or 2 mg/l mL, or 3 mg/l mL, or 4 mg/l mL, or 5 mg/l mL, or 6 mg/l mL, or 7 mg/l mL, or 8 mg/l mL, or 9 mg/l mL, or 10 mg/l mL, or 15 mg/l mL, or 20 mg/l mL, or 25 mg/l mL, or 30 mg/l mL, or 35 mg/l mL, or 40 mg/l mL. The microinjection device can be used to deliver ondansetron to a subject over a predetermined time period, such as a time period up to and including 1 minute, or 2 minutes, or 3 minutes, or 4 minutes, or 5 minutes, or 6 minutes, or 7 minutes, or 8 minutes, or 9 minutes, or 10 minutes, or 20 minutes, or 30 minutes, or 40 minutes, or 50 minutes, or 1 hour, or 2 hours, or 3 hours, or 4 hours, or 5 hours, or 6 hours, or 7 hours, or 8 hours, or 9 hours, or hours, or 11 hours, or 12 hours, or 13 hours, or 14 hours, or 15 hours, or 16 hours, or 17 hours, or 18 hours, or 19 hours, or 20 hours, or 21 hours, or 22 hours, or 23 hours, or 24 hours. For example, a subject can apply a microinjection device having an ondansetron formulation with an ondansetron concentration of about 8 mg/l mL to the subject's arm for delivery of ondansetron on a daily basis.

In an embodiment, the equivalent of 0.001 mg, or 0.002 mg, or 0.003 mg, or 0.004 mg, or 0.005 mg, or 0.006 mg, or 0.007 mg, or 0.008 mg, or 0.009 mg, or 0.01 mg, or 0.02 mg, or 0.03 mg, or 0.04 mg, or 0.05 mg, or 0.06 mg, or 0.07 mg, or 0.08 mg, or 0.09 mg, or 0.1 mg, or 0.15 mg, or 0.2 mg, or 0.25 mg, or 0.3 mg, or 0.35 mg, or 0.4 mg, or 0.45 mg, or 0.5 mg, or 0.55 mg, or 0.6 mg, or 0.65 mg, or 0.7 mg, or 0.75 mg, or 0.8 mg, or 0.85 mg, or 0.9 mg, or 0.95 mg, or 1 mg of palonosetron in a 1 mL solution is delivered to a subject once a day. In another embodiment, 0.001 mg, or 0.002 mg, or 0.003 mg, or 0.004 mg, or 0.005 mg, or 0.006 mg, or 0.007 mg, or 0.008 mg, or 0.009 mg, or 0.01 mg, or 0.02 mg, or 0.03 mg, or 0.04 mg, or 0.05 mg, or 0.06 mg, or 0.07 mg, or 0.08 mg, or 0.09 mg, or 0.1 mg, or 0.15 mg, or 0.2 mg, or 0.25 mg, or 0.3 mg, or 0.35 mg, or 0.4 mg, or 0.45 mg, or 0.5 mg, or 0.55 mg, or 0.6 mg, or 0.65 mg, or 0.7 mg, or 0.75 mg, or 0.8 mg, or 0.85 mg, or 0.9 mg, or 0.95 mg, or 1 mg of palonosetron in a 1 mL solution is delivered to a subject once a day and over a time period up to and including 1 minute, or 2 minutes, or 3 minutes, or 4 minutes, or 5 minutes, or 6 minutes, or 7 minutes, or 8 minutes, or 9 minutes, or 10 minutes, or 20 minutes, or 30 minutes, or 40 minutes, or 50 minutes, or 1 hour, or 2 hours, or 3 hours, or 4 hours, or 5 hours, or 6 hours, or 7 hours, or 8 hours, or 9 hours, or 10 hours, or 11 hours, or 12 hours, or 13 hours, or 14 hours, or 15 hours, or 16 hours, or 17 hours, or 18 hours, or 19 hours, or 20 hours, or 21 hours, or 22 hours, or 23 hours, or 24 hours. In an embodiment, a microinjection device, such as any device provided herein, is loaded with a palonosetron formulation having a concentration of about 1 mg/l mL, or 2 mg/l mL, or 3 mg/l mL, or 4 mg/l mL, or 5 mg/l mL, or 6 mg/l mL, or 7 mg/l mL, or 8 mg/l mL, or 9 mg/l mL, or 10 mg/l mL, or 15 mg/l mL, or 20 mg/l mL, or 25 mg/l mL, or 30 mg/l mL, or 35 mg/l mL, or 40 mg/l mL. The microinjection device can be used to deliver palonosetron to a subject over a predetermined time period, such as a time period up to and including 1 minute, or 2 minutes, or 3 minutes, or 4 minutes, or 5 minutes, or 6 minutes, or 7 minutes, or 8 minutes, or 9 minutes, or 10 minutes, or 20 minutes, or 30 minutes, or 40 minutes, or 50 minutes, or 1 hour, or 2 hours, or 3 hours, or 4 hours, or 5 hours, or 6 hours, or 7 hours, or 8 hours, or 9 hours, or 10 hours, or 11 hours, or 12 hours, or 13 hours, or 14 hours, or 15 hours, or 16 hours, or 17 hours, or 18 hours, or 19 hours, or 20 hours, or 21 hours, or 22 hours, or 23 hours, or 24 hours. For example, a subject can apply a microinjection device having a palonosetron formulation with a palonosetron concentration of about 0.05 mg/l mL to the subject's arm for delivery of palonosetron on a daily basis.

In an embodiment, a microinjection device having a deliverable serotonin receptor antagonist formulation is provided. A serotonin receptor antagonist, ondansetron formulation or palonosetron formulation can be delivered by subcutaneous, transdermal or intradermal injection. A user places the device adjacent another user's skin or adjacent the user's skin, if self administration is desired, to deliver the serotonin receptor antagonist formulation. The user employs the microinjection device to deliver the serotonin receptor antagonist formulation to another user or the user (self administration). The user then removes the microinjection device from the skin. In an embodiment, the microinjection device is a single use device and is be disposed of after it is used. In another embodiment the microinjection device can be used for a future administration of the serotonin receptor antagonist formulation, such as with a replaceable cartridge or with additional doses provided in the original cartridge having the serotonin receptor antagonist formulation.

In various embodiments, a formulation comprising a plurality of serotonin receptor antagonists can be administered to a subject with the aid of a microinjection device. In an embodiment, a formulation comprising ondansetron and palonosetron can be administered to a subject with the aid of a microinjection device. The dosages of each of serotonin receptor antagonist in the formulation can be selected as desired for the purposes of therapy or treatment. In an embodiment, a serotonin receptor antagonist formulation can include ondansetron at a concentration of about 8 mg/l mL or palonosetron at a concentration of about 0.05 mg/l mL. In some cases, a serotonin receptor antagonist formulation can include ondansetron at a concentration of at least about 8 mg/l mL and palonosetron at a concentration of at least about 0.05 mg/l mL.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1.-38. (canceled)

39. A microinjection device having a serotonin receptor antagonist formulation, said microinjection device configured to deliver said serotonin receptor antagonist formulation to a subject.

40. The microinjection device of claim 39, wherein the serotonin receptor antagonist formulation comprises ondansetron, palonosetron, tropisetron, granisetron, dolasetron, metoclopramide, benzoylmethylecgonine, or a pharmaceutically acceptable derivative thereof.

41. The microinjection device of claim 39, wherein the serotonin receptor antagonist formulation has a serotonin receptor antagonist concentration between about 0.001 mg in 1 milliliter (mL) and 40 mg in 1 mL.

42. The microinjection device of claim 41, wherein the serotonin receptor antagonist concentration is between about 0.01 mg in 1 mL and 30 mg in 1 mL.

43. The microinjection device of claim 39, wherein the serotonin receptor antagonist formulation has a pH between about 2.0 and 6.0.

44. The microinjection device of claim 43, wherein the serotonin receptor antagonist formulation has a pH between about 3.0 and 5.9.

45.-55. (canceled)

56. A system for the administration of a serotonin receptor antagonist to a subject, comprising:
a serotonin receptor antagonist formulation; and
a microinjection device.

57. The system of claim 56, wherein the serotonin receptor antagonist formulation is ondansetron, palonosetron, tropisetron, granisetron, dolasetron, metoclopramide, benzoylmethylecgonine, or a pharmaceutically acceptable derivative thereof.

58. The system of claim 56, wherein the serotonin receptor antagonist formulation has a serotonin receptor antagonist concentration between about 0.001 mg in 1 milliliter (mL) and 40 mg in 1 mL.

59. The system of claim 58, wherein the serotonin receptor antagonist concentration is between about 0.01 mg in 1 mL and 30 mg in 1 mL.

60. The system of claim 56, wherein the serotonin receptor antagonist formulation has a pH between about 2.0 and 6.0.

61. The system of claim 60, wherein the serotonin receptor antagonist formulation has a pH between about 3.0 and 5.9.

62.-105. (canceled)

106. A method for subcutaneous, transdermal or intradermal delivery of a serotonin receptor antagonist formulation to a subject, comprising: administering the serotonin receptor antagonist formulation to the subject by microneedle injection, hydration, ablation of the subject's skin, follicular delivery, ultrasound, iontophoresis or electroporation.

107. The method of claim 106, wherein the serotonin receptor antagonist formulation comprises ondansetron, palonosetron, tropisetron, granisetron, dolasetron, metoclopramide, benzoylmethylecgonine, or a pharmaceutically acceptable derivative thereof.

108. The method of claim 106, wherein the serotonin receptor antagonist formulation has a serotonin receptor antagonist concentration between about 0.001 mg in 1 milliliter (mL) and 40 mg in 1 mL.

109. The method of claim 108, wherein the serotonin receptor antagonist formulation has a serotonin receptor antagonist concentration between about 0.01 mg in 1 mL and 30 mg in 1 mL.

110. The method of claim 106, wherein the serotonin receptor antagonist formulation has a pH between about 2.0 and 6.0.

111. The method of claim 110, wherein the serotonin receptor antagonist formulation has a pH between about 3.0 and 5.9.

112.-113. (canceled)

114. The method of claim 106, wherein the serotonin receptor antagonist formulation is administered to the subject by microneedle injection.

115.-143. (canceled)

144. The system of claim 39, wherein the serotonin receptor antagonist formulation comprises a pharmaceutically acceptable excipient.

145. The system of claim 56, wherein the serotonin receptor antagonist formulation comprises a pharmaceutically acceptable excipient.

146. The system of claim 106, wherein the serotonin receptor antagonist formulation comprises a pharmaceutically acceptable excipient.

147. The microinjection device of claim 39, wherein said microinjection device comprises:
a housing having a skin-contacting face defining an opening that can be positioned at a target site, said housing having a microneedle array; and
an impactor for impacting the microneedle array and accelerating the microneedle array toward the target site, the microneedle array configured to deliver the serotonin receptor antagonist formulation to the subject,
wherein the impactor is configured to move along a substantially arcuate path to move the microneedle array toward the target site.

148. The system of claim 56, wherein said microinjection device comprises:
a housing having a skin-contacting face defining an opening that can be positioned at a target site, said housing having a microneedle array; and
an impactor for impacting the microneedle array and accelerating the microneedle array toward the target site, the microneedle array configured to deliver the serotonin receptor antagonist formulation to the subject,
wherein the impactor is configured to move along a substantially arcuate path to move the microneedle array toward the target site.

149. The method of claim 114, wherein microneedle injection comprises using a microinjection device to deliver the serotonin receptor antagonist formulation to the subject, wherein said microinjection device comprises:
a housing having a skin-contacting face defining an opening that can be positioned at a target site, said housing having a microneedle array; and
an impactor for impacting the microneedle array and accelerating the microneedle array toward the target site, the microneedle array configured to deliver the serotonin receptor antagonist formulation to the subject,
wherein the impactor is configured to move along a substantially arcuate path to move the microneedle array toward the target site.
